(19) United States
(12) Patent Application Publication
(10) Pub. No.: US 2002/0042054 A1
(43) Pub. Date: Apr. 11, 2002

(54) Vigilance nucleic acids and related diagnostic, screening and therapeutic methods

(76) Inventors: Giulio Tononi, Escondido, CA (US); Chiara Cirelli, Escondido, CA (US); Paul J. Shaw, San Diego, CA (US); Ralph J. Greenspan, Coronado, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/733,607

(22) Filed: Dec. 8, 2000

Related U.S. Application Data
(60) Provisional application No. 60/304,200, filed on Dec. 8, 1999.

(51) Int. Cl. C12Q 1/68; C07H 21/04; A61K 31/00

(52) U.S. Cl. 435/6; 514/1; 536/23.2

(57) ABSTRACT

The invention provides novel isolated vigilance nucleic acid molecules, and kits containing two or more isolated vigilance nucleic acid molecules. Methods for diagnosing and treating vigilance disorders, for determining and altering vigilance levels, and for screening for therapeutic compounds useful for treating vigilance disorders and altering vigilance level, are also provided. The diagnostic, therapeutic and screening methods of the invention involve determining expression or activity profiles of vigilance genes.

This application claims the benefit of U.S. Provisional Application No. 60/______ (yet to be assigned), filed Dec. 8, 1999, which was converted from U.S. Ser. No. 09/456,785, and is incorporated herein by reference.

### BACKGROUND OF THE INVENTION

Sleep is a naturally occurring, periodic, reversible state of unconsciousness that is ubiquitous in mammals and birds, although its precise function is not known. The importance of sleep is suggested by its homeostatic regulation: the longer an animal is awake, the more it needs to sleep.

In humans, obtaining less than the required number of hours of sleep, particularly over several nights, leads to a decreased ability to retain new information, impaired productivity, altered mood, lowered resistance to infection and an increased susceptibility to accidents. Sleep-related traffic accidents annually claim thousands of lives, and operator fatigue has also been shown to play a contributory role in airplane crashes and other catastrophic accidents.

Besides lifestyle factors, a variety of physiological and psychological disorders can affect sleep patterns. The most common sleep disorder is primary insomnia, or a difficulty in initiating or maintaining sleep, which affects a large percentage of the population at some point in their lives. Other common sleep disorders include hypersomnia, or excessive daytime sleepiness, and narcolepsy, which is characterized by sudden and irresistible bouts of sleep.

Currently available drugs used to modulate vigilance, such as drugs that induce sleep, prolong wakefulness, or enhance alertness, suffer from a number of shortcomings. For example, available sleep-inducing drugs often do not achieve the fully restorative effects of normal sleep. Often such drugs cause undesirable effects upon waking, such as anxiety or continued sedation. Many available drugs that increase vigilance do so with a characteristic “crash” when the effect of the drugs wears off. Furthermore, many of the currently available drugs that modulate sleep and wakefulness are addictive or have adverse effects on learning and memory.

Clearly, there is a need to identify drugs that induce restorative sleep or that increase vigilance, without undesirable side effects. Unfortunately, current methods for screening for such drugs, using mammals, are slow, burdensome and expensive. Thus, there exists a need for improved methods for screening for drugs that modulate sleep and vigilance.

Sleep disorders are very common, yet often go undiagnosed or misdiagnosed because the molecular correlates of these disorders are poorly understood. Additionally, drugs that alter vigilance in normal individuals and individuals suffering from vigilance disorders may not be effective, or may have undesirable side effects, because the drug does not target the relevant genes or gene products that regulate the vigilance state or mediate the vigilance disorder.

Thus, there also exists a need to identify genes whose expression or activity is associated with vigilance level or with particular vigilance disorders. Identification of such genes and their expression and activity profiles would allow more accurate diagnosis of vigilance disorders and more accurate and rapid determination of vigilance levels. Identification of such genes also provides rapids methods of identifying therapeutic agents that specifically modulate the expression or activity of the relevant genes associated with vigilance. Such therapeutic agents can be used to effectively treat vigilance disorders or to appropriately alter vigilance levels or states in normal individuals.

The present invention satisfies these needs and provides related advantages as well.

### SUMMARY OF THE INVENTION

The invention provides a method of identifying a compound that alters vigilance. The method consists of contacting an invertebrate with a candidate compound, evaluating a vigilance property in the contacted invertebrate, and determining if the candidate compound alters the vigilance property in the contacted invertebrate. A candidate compound that alters the vigilance property in the contacted invertebrate is identified as a compound that alters vigilance.

In one embodiment, the vigilance property evaluated is a behavioral property, including activity, latency to sleep or arousal threshold. In another embodiment, the vigilance property evaluated is a molecular property, including expression of one or more vigilance-modulated genes.

The invention also provides a method of identifying a vigilance enhancing compound that modulates homeostatic regulation. The method consists of contacting an invertebrate with a compound that increases vigilance, and determining the effect of the compound on a homeostatic regulatory property of vigilance. A compound that alters the homeostatic regulatory property is characterized as being a vigilance enhancing compound that modulates homeostatic regulation.

Also provided is a method of identifying a vigilance diminishing compound that modulates homeostatic regulation. The method consists of contacting an invertebrate with a compound that decreases vigilance, and determining the effect of the compound on a homeostatic regulatory property of vigilance. A compound that alters the homeostatic regulatory property is characterized as being a vigilance diminishing compound that modulates homeostatic regulation.

The invention further provides an isolated vigilance nucleic acid molecule, containing a nucleotide sequence selected from the group consisting of SEQ ID NOS:1-6 and 8-27, or modification thereof. Further provided is an isolated oligonucleotide, containing at least 15 contiguous nucleotides of the nucleotide sequence of SEQ ID NOS:1-6 and 8-27, or the antisense strand thereof. Also provides are kit containing two or more isolated vigilance nucleic acid molecules or oligonucleotides. The vigilance nucleic acid molecules and olignucleotides can be optionally attached to a solid support.

Also provided is a method of diagnosing a vigilance disorder in an individual. The method consists of determining a vigilance gene profile of the individual, and comparing the profile to a control profile indicative of the vigilance disorder. Correspondence between the profile of the individual and the control profile indicates that said individual has the vigilance disorder. Further provided is a method of determining vigilance level in an individual. The method consists of determining a vigilance gene profile of the individual, and comparing the profile to a control profile indicative of a predetermined vigilance level. Correspondence between the profile of the individual and the control profile indicates that the individual exhibits said vigilance level. In such methods, at least one vigilance gene profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene comprising a nucleotide sequence of any of SEQ ID NOS:2-6, 8-14 and 16-27 or modification thereof.

The invention also provides a method of determining the efficacy of a compound in ameliorating a vigilance disorder. The method consists of administering the compound to an individual having a vigilance disorder, and determining an effect of the compound on the vigilance gene profile of the individual. Modulation of the vigilance gene profile of the individual to correspond to a normal vigilance profile indicates that the compound is effective in ameliorating the vigilance disorder. The invention further provides a method of determining the efficacy of a compound in modulating vigilance. The method consists of administering the compound to an individual, and determining an effect of the compound on the vigilance gene profile of the individual. Modulation of the vigilance gene profile indicates that the compound modulates vigilance. In such methods, at least one vigilance gene profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene comprising a nucleotide sequence of any of SEQ ID NOS:2-6, 8-14 and 16-27 or modification thereof.

The invention further provides a method of ameliorating a vigilance disorder in an individual. The method consists of administering to an individual having a vigilance disorder an agent that modulates the vigilance gene profile of the individual to correspond to a normal vigilance gene profile. The invention also provides a method of modulating vigilance level in an individual. The method consists of administering to an individual an agent that modulates the vigilance gene profile of the individual to correspond to a control vigilance gene profile. In such methods, at least one vigilance gene profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene comprising a nucleotide sequence of any of SEQ ID NOS:2-6, 8-14 and 16-27 or modification thereof.

### BRIEF DESCRIPTION OF THE DRAWINGS

FIG. 1A shows a schematic of the ultrasound activity monitoring system. FIG. 1B shows a trial comparing Drosophila activity detected by the ultrasound apparatus (gray columns) to three behavioral states scored by a human observer (black lines). FIG. 1C shows Drosophila activity during the light period (horizontal white bar) and the dark period (horizontal black bar).

FIG. 2 shows the rest-activity system monitored in 5-day old female flies using the infrared system. FIG. 2A shows amount of rest under base-line conditions (open circles), following manual rest-deprivation during the dark period (black squares), and following automated rest-deprivation during the dark period (gray triangles). FIG. 2B shows amount of rest under base-line conditions (open circles) and following automated rest-deprivation during the light period (gray triangles). FIG. 2A Inset shows rest under constant darkness in control per⁰¹ flies (open circles) and in rest-deprived per⁰¹ flies (black squares). FIG. 2B Inset shows a plot of rest during recovery versus activity during rest deprivation.

FIGS. 3A and 3B show rest as a function of Drosophila age for a 24-hour period. Rest during the light period (horizontal white bar) and the dark period (horizontal black bar) for flies 1 day after eclosion (black squares), 2 days after eclosion (gray triangles), 3 days after eclosion (open circles), 16 days after eclosion (gray diamonds), and 33 days after eclosion (black circles) is shown. FIG. 3C shows rest during dark period in Drosophila given the indicated doses of caffeine beginning in the final hour of the light period. FIG. 3D shows rest in the first hour of the dark period, and FIG. 3E shows latency to first dark rest, in Drosophila given the indicated doses of hydroxyzine beginning in the final hour of the light period.

FIG. 4A shows the three experimental conditions used to evaluate changes in gene expression, waking (W), rest (R) and rest deprivation (RD). White bars indicate the light period, black bars indicate the dark period. The graphs in FIGS. 4B-4D show densitometric analysis of mRNA levels of vigilance-modulated genes evaluated using ribonuclease protection assays. FIG. 4B shows levels of Fas and Cyp4e2 mRNA in flies. FIG. 4C shows levels of Cytochrome oxidase C subunit I mRNA in flies and rats. FIG. 4D shows levels of BiP in flies and rats.

FIG. 5A shows the number of infrared beam crossings per day in wild-type, Dat^{lo}/Dat^{lo} and Dat^{lo}/Df flies (p>0.05, n=25). FIG. 5B shows activity patterns as measured by the ultrasound system in wild-type, Dat^{lo}/Dat^{lo} and Dat^{lo}/Df flies (representative activity records for 1 h during the light period are shown). FIG. 5C rest rebound in wild-type, Dat^{lo}/Dat^{lo} and Dat^{lo}/Df flies during the first 6 h of recovery. FIG. 5D shows rest rebound in wild-type Dat^{lo}/Dat^{lo} and Dat^{lo}/Df flies during the second 6 h of recovery.

### DETAILED DESCRIPTION OF THE INVENTION

The present invention provides methods of rapidly and efficiently identifying compounds that alter vigilance, including compounds that promote sleep, prevent sleep, or increase vigilance. The compounds identified by the methods of the invention can thus be used to treat individuals suffering from psychological, physiological or genetic conditions that deprive them of restorative sleep or that cause excessive sleepiness. These compounds can also be used to prolong wakefulness, such as when it is desired to extend an individual's productivity, or to increase attentiveness, learning or memory.

Sleep in mammals has been defined by several criteria, including electrophysiological and behavioral criteria. Behavioral criteria for sleep include sustained quiescence, increased arousal threshold, and “sleep rebound,” or increased sleep or increased sleep intensity following prolonged waking. The criterion of sleep rebound indicates that sleep is under homeostatic control and is thus distinguishable from mere inactivity.

Recently, physiological correlates of sleep in mammals have been extended to the level of gene expression. Molecular screening has revealed that brain levels of mitochondrial enzymes and of several genes implicated in neural plasticity are high during waking and low during sleep (see, for example, (see Cirelli et al., Mol. Brain Res. 56:293 (1998); Cirelli et al., Ann. Med. 31:117 (1999); and Cirelli et al., Sleep 22(S):113 (1999)). Therefore, sleep in mammals can also be characterized by a distinct pattern of gene expression.

Although it is well-known that most organisms exhibit circadian rest-activity cycles, prior to the present invention it was not known that invertebrates exhibit a sleep-like state that is comparable, by behavioral, physiological, developmental, molecular and genetic criteria, to mammalian sleep. This sleep-like state in invertebrates is henceforth referred to as “sleep.”

As described herein, invertebrate sleep is very similar, by behavioral criteria, to mammalian sleep. More specifically, as shown in Example I, sleep in an exemplary invertebrate,Drosophila melanogaster, is associated with sustained behavioral quiescence and increased arousal threshold. Additionally, sleep deprivation during the normal sleep period led to a rebound effect comparable to sleep rebound in mammals, indicating that sleep is under similar homeostatic control in invertebrates.

Furthermore, as described herein, sleep in invertebrates is dependent on age, and follows a similar pattern of age dependency as mammalian sleep, indicating that sleep in invertebrates is developmentally regulated. Likewise, sleep remains homeostatically regulated in older invertebrates, as it is in older mammals (see Example II). Additionally, sleep and wake in invertebrates are subject to pharmacological manipulation using compounds that are known to act as stimulants or hypnotics in mammals (see Example III).

Furthermore, of importance to the determination that sleep and wake in invertebrates are truly similar to mammalian sleep and wake, it is also described herein that several classes of genes, and several individual genes, whose regulation is dependent on vigilance state in mammals are similarly regulated in invertebrates (see Example IV). Additionally, as disclosed herein, mutations in genes that regulate sleep in invertebrates affect vigilance properties, including homeostatic regulation of sleep (see Example IV). Likewise, mutations have been identified in mammalian genes that affect sleep, including orexin (see Chemelli et al., Cell 98:437-451 (1999)), indicating that in both invertebrates and mammals, vigilance is under genetic control.

The discovery that invertebrates exhibit sleep and wake states that are similar by behavioral, developmental, pharmacological, genetic and molecular criteria to mammalian sleep and wake, provides a basis for the methods disclosed herein of identifying novel compounds that can be used to modulate vigilance in mammals by screening compounds for their effect on vigilance properties in invertebrates.

The invention provides a method of identifying a compound that alters vigilance. The method consists of contacting an invertebrate with a candidate compound, evaluating a vigilance property in the contacted invertebrate, and determining if the candidate compound alters the vigilance property in the contacted invertebrate. A candidate compound that alters the vigilance property in the contacted invertebrate is identified as a compound that alters vigilance.

As used herein, the term “vigilance” is intended to mean the degree or extent to which an organism exhibits sleep or wake behaviors. Thus, the term “altering vigilance” is intended to encompass a change in state from wake to sleep or vice-versa, as well as any increase or decrease in intensity or duration of behaviors associated with a sleep or wake state.

The methods of the invention can be used to identify compounds that either increase or decrease vigilance. A compound that increases vigilance can, for example, cause the animal to wake from sleep, prolong periods of wakefulness, prolong normal latency to sleep, restore normal sleep patterns following sleep deprivation, or enhance beneficial wake-like characteristics, such as alertness, responsiveness to stimuli, energy, and ability to learn and remember. In contrast, a compound that decreases vigilance can, for example, cause an animal to sleep, prolong periods of sleep, promote restful sleep, decrease latency to sleep, or decrease unwanted wake-like characteristics, such as anxiety and hyperactivity.

As used herein, the term “vigilance property” is intended to mean a behavioral, physiological or molecular property in invertebrates that is correlated with mammalian sleep and wake states. As described further below, invertebrates can exhibit a variety of behavioral properties that are closely correlated with mammalian sleep and wake states, including activity, arousal threshold and latency to sleep. Additionally, as described further below, invertebrates can exhibit a variety of molecular properties that are closely correlated with mammalian sleep and wake states, including expression of vigilance-modulated genes. Invertebrates can also exhibit physiological properties that are closely correlated with mammalian sleep, including the frequency, type and intensity of neuronal signals, heart rate, and the like.

Generally, invertebrates exhibit circadian patterns of rest and activity, with most rest occurring during the night in diurnal animals and most activity occurring during the day. In contrast, in nocturnal animals most rest occurs during the day, whereas most activity takes place during the night. Under laboratory conditions, it is possible to regulate the circadian rest-activity cycle by regulating the length of light and dark, and thus establish what are referred to herein as “normal wake periods” and “normal sleep periods.” For example, in Drosophila melanogaster subjected to a 12 h:12 h light:dark cycle, the “normal wake period” is the 12 hour light period, whereas the “normal sleep period” is the 12 hour dark period. Those skilled in the art can readily determine or establish normal wake and sleep periods for other invertebrates.

An example of a behavioral vigilance property that can be evaluated in invertebrates is activity during all or part of a normal wake or sleep period. As used herein, the term “activity” is intended to encompass all behavioral activities normally exhibited by that invertebrate including, for example, locomoting, movements of body parts, grooming, eating, and the like, in contrast to “inactivity” or “rest.” Activity can be evaluated throughout a normal wake period or throughout a normal sleep period, or both, or evaluated for only part of a normal wake or sleep period, such as for at least 10 minutes, 30 minutes, 1, 2, 4, 6, 8 or 12 hours. Once activity during a normal sleep period or normal wake period is established, those skilled in the art can readily evaluate whether a candidate compound increases or decreases intensity of activity or alters the pattern of activity during all or part of that period.

For certain applications of the method, it will be preferable to evaluate activity following sleep deprivation. As described previously, sleep rebound following sleep deprivation is a characteristic of homeostatically regulated sleep. Thus, by establishing the normal sleep rebound behavior of the invertebrate, those skilled in the art can readily evaluate whether a candidate compound affects the normal homeostatic regulation of sleep.

As used herein, the term “sleep deprivation” refers to depriving the animal of rest. This deprivation is generally for a sufficient period of time during a normal sleep period to result in a detectable decrease in activity, increase in sleep, or increase in intensity of sleep during the subsequent period, also known as a “sleep rebound” effect. In general, sleep deprivation results from depriving the animal of rest during at least 10%, such as at least 25%, including from 50% to 100% of the normal sleep period.

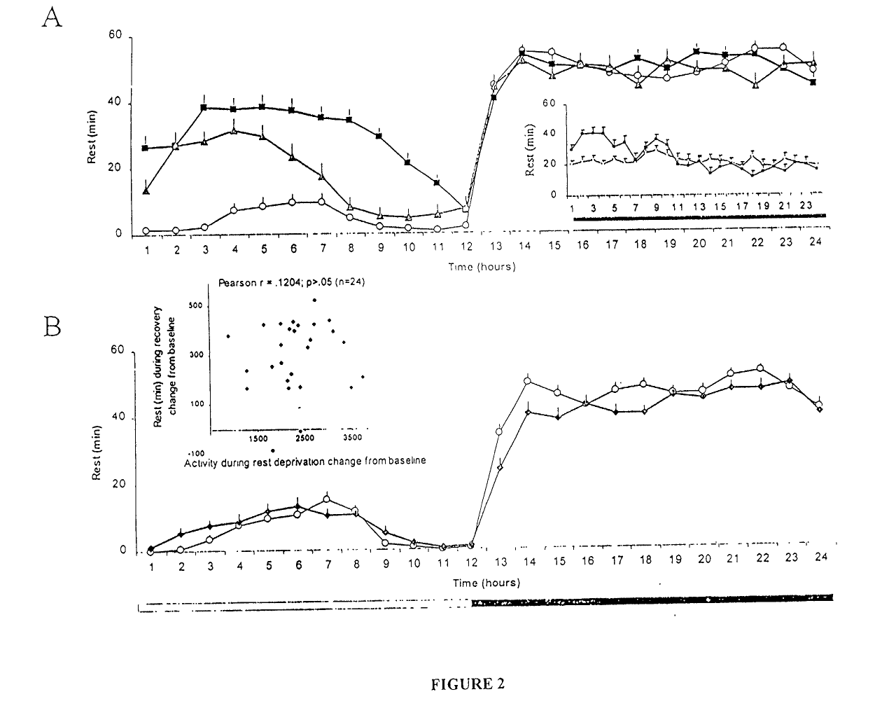

Any method appropriate for the particular invertebrate can be used to deprive an animal of sleep. As described in Example I, Drosophila melanogaster can be sleep-deprived for the entire normal sleep period, using manual or automated physical stimulation, and the amount, pattern and intensity of activity indicative of sleep rebound evaluated (see FIG. 2A). In other organisms, it may be preferable to sleep-deprive the animals using electrical stimulation, noise, or other stimuli, for longer or shorter periods. The time period and method for sleep-depriving an animal can be determined by those skilled in the art for a particular application.

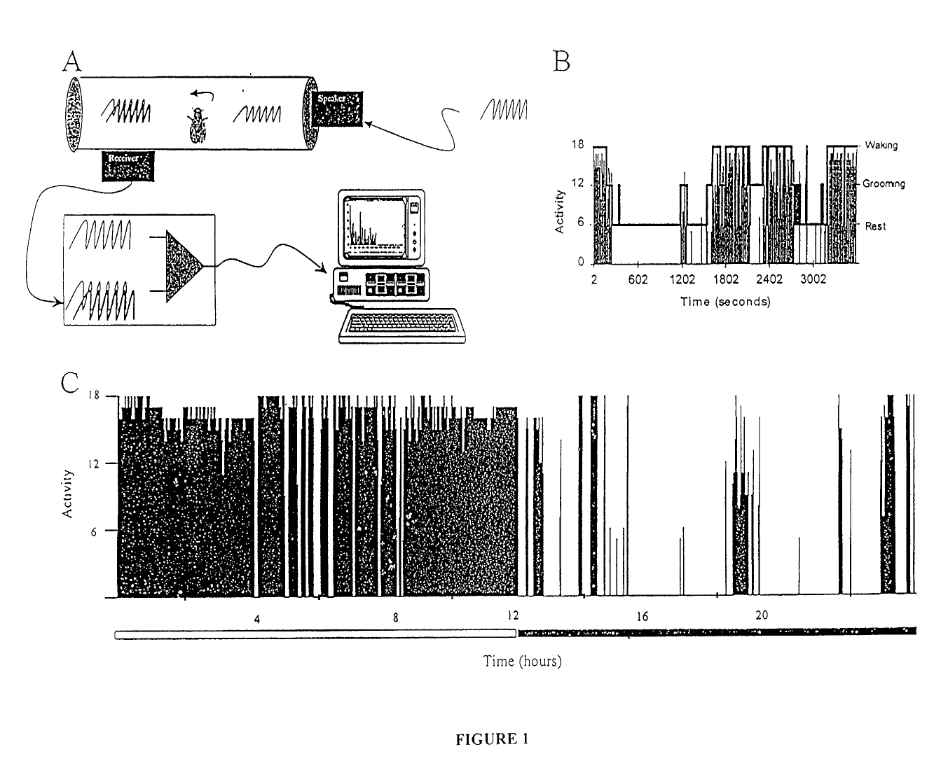

Various manual and automated assays can be used to evaluate intensity and patterns of activity. For example, activity can be detected visually, either by direct observation or by time-lapse photography. Alternatively, an ultrasound monitoring system can be used, such as the system shown in FIG. 1A and described in Example I, below. Such a system is advantageous in detecting very small movements of the animals' body parts and, as shown in FIG. 1B, the output is closely correlated with visual observations. An example of the activity of Drosophila melanogaster during a normal wake period (12 hour light period) and a normal sleep period (12 hour dark period), as evaluated using an ultrasound monitoring system, is shown in FIG. 1C.

As a further example, an infrared monitoring system, such as the infrared Drosophila Activity Monitoring System available from Trikinetics (described in M. Hamblen et al., J. Neuroqen. 3:249 (1986)), can be used. As described in Example I, below, an infrared monitoring system is advantageous when simultaneously evaluating activity in large numbers of invertebrates. An example of the activity of a population of Drosophila melanogaster during a normal wake period (12 hour light period) and a normal sleep period (12 hour dark period), as evaluated using an ultrasound monitoring system, is shown in FIG. 1C.

Those skilled in the art can determine an appropriate method to evaluate invertebrate activity in a particular application of the method, depending on considerations such as the size and number of invertebrates, their normal activity level, the intended number of data points, and whether a quantitative or qualitative assessment of activity is desired.

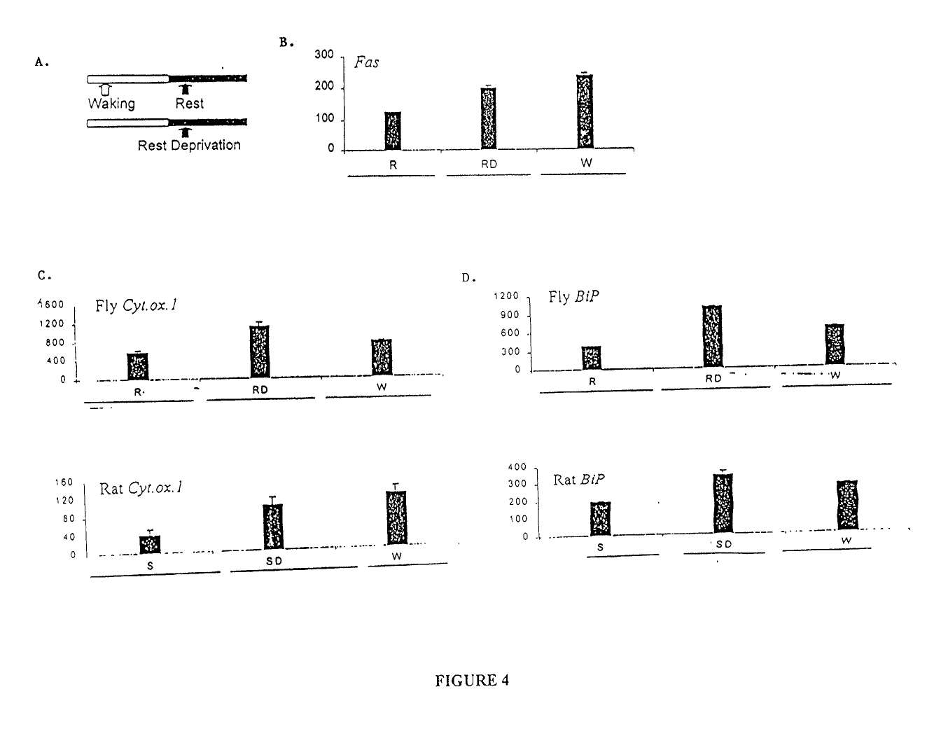

A further example of a behavioral vigilance property that can be evaluated in invertebrates is latency to sleep. As used herein, the term “latency to sleep” refers to the period of time to the first rest bout following the change from the normal wake period to the normal sleep period (ie. from light to dark in diurnal animals, or from dark to light in nocturnal animals). As shown in FIG. 4E, latency to sleep in control Drosophila melanogaster was about 40 minutes. If desired, latency to sleep following sleep deprivation can also be established. Once normal latency to sleep, or latency to sleep following sleep deprivation are established for a particular invertebrate, one skilled in the art can evaluate whether a candidate compound increases or decreases this vigilance property.

Another example of a behavioral vigilance property that can be evaluated in invertebrates is arousal threshold. As used herein, the term “arousal threshold” refers to the amount of stimulation required to elicit a behavioral response, such as movement. Any reproducible stimulus can be used to evaluate arousal threshold including, for example, vibratory stimulus, noise, electrical stimulation, heat, or light.

Invertebrates that are in a wake state will exhibit a behavioral response at a lower level of stimulation than invertebrates that are in a sleep state. For example, as described in Example I, below, when subjected to vibratory stimuli of varying intensities, Drosophila melanogaster that were in a wake-like state, as determined by activity criteria, responded to low-level stimuli that did not elicit a response in flies that were in a sleep state. Furthermore, an animal that is deeply asleep will exhibit an increased arousal threshold compared to an animal that less deeply asleep. Accordingly, arousal threshold is a measure of sleep versus wake, as well as intensity of sleep. Once normal arousal threshold associated with sleep and wake are established for a particular invertebrate, those skilled in the art can readily evaluate whether a candidate compound increases or decreases this vigilance property.

Other vigilance properties that can be measured in invertebrates include molecular properties correlated with sleep and wake states. As used herein, the term “molecular property” refers to any property that can be evaluated in invertebrate tissues, cells or extracts, including, for example, production or turnover of a second messengers, GTP hydrolysis, influx or efflux of ions or amino acids, membrane voltage, protein phosphorylation or glycosylation, membrane voltage, enzyme activity, protein-protein interactions, protein secretion, and gene expression.

A specific example of a molecular vigilance property that can be evaluated in invertebrates is expression of one or more vigilance-modulated genes. As used herein, the term “expression” is intended to encompass expression at the mRNA or polypeptide level. Accordingly, expression of a vigilance-modulated gene can be evaluated by any qualitative or quantitative method that detects mRNA, protein or activity, including methods described further below. Once the abundance or pattern of expression of vigilance-modulated genes are established for a particular invertebrate, those skilled in the art can readily evaluate whether a candidate compound increases or decreases expression of one or more vigilance-modulated genes.

As used herein, the term “vigilance-modulated gene” refers to a gene whose expression level varies according to vigilance state. For example, the expression level of a vigilance-modulated gene can normally vary by at least about 10%, such as at least 25%, or at least about 50%, including at least about 100%, 250%, 500%, 1000% more between sleep and wake. As described herein, at least about 1% of the transcripts in invertebrates are modulated by vigilance state and, consequently, correspond to vigilance-modulated genes. Therefore, in the methods of the invention one can evaluate expression of at least one vigilance-modulated gene, such as at least 2, 5, 10, 20, 50, 100 or more vigilance-modulated genes. Although not necessary for the practice of the invention, as described below, these genes can be cloned and/or their sequences determined using standard molecular biology procedures.

If desired for a particular application of the method, genes whose expression is normally upregulated in the wake-like state, or genes whose expression is normally upregulated in sleep, or any combination, can be evaluated.

Exemplary vigilance-modulated genes identified in Drosophila melanogaster, with their sequence identifiers or GenBank Accession Nos. in brackets, and the GenBank Accession Nos. of their apparent rat or human homologs, are as follows: an apparent homolog of mammalian Fatty acid synthase (Fas) (contains SEQ ID NO:1; human:NM_{—}004104); Cytochrome oxidase C, subunit I (mt:Co1) (J01404, J01405, and J01407; rat:J01435); Cytochrome p450 (Cyp4e2) (X86076; rat:U39206; human:AF054821)); BiP (also known as Hsc70-3) (L01498; contains SEQ ID NO:7; human:AF188611); and arylalkyamine N-acetyl transferase (Dat) (Y07964; human:NM_{—}001088). Each of these genes was expressed at higher levels during waking than during sleep (see Example IV). In contrast, a gene designated “Rest” was 45% higher during sleep than during rest.

Other Drosophila genes that are upregulated during wake contain the nucleotide sequences designated SEQ ID NOS:4, 5 and 6. Other Drosophila genes that are upregulated during sleep contain the nucleotide sequences designated SEQ ID NOS:2 and 3.

As disclosed herein, there is similarity between vigilance-modulated gene expression in rats and in Drosophila melanogaster, both in terms of number and type of genes that are modulated. For example, as described in Example IV, below, Cytochrome oxidase C, subunit I shows a rapid increase in expression during the first few hours of waking in both rats and Drosophila. Likewise, expression of a Drosophila and a rat Cytochrome P450 (U39206, U39207) were similarly upregulated in waking and sleep deprivation. Therefore, vigilance-modulated genes in invertebrates include homologs of genes whose expression levels vary with the vigilance state of mammals.

A variety of vigilance-modulated genes in rats are described in Cirelli et al., Mol. Brain Res. 56, 293 (1998); Cirelli et al., Ann. Med. 31:117 (1999); Cirelli et al., Sleep 22(S):113 (1999) and include the following genes, with their GenBank Accession Numbers given in brackets: immediate-early genes, transcription factors and chaperones (e.g. NGFI-A (M18416), NGFI-B (U17254), Zn-15 related zinc finger (rlf; U22377), Arc (U19866), JunB (X54686) and IER5 (AW142256)); mitochondrial genes (e.g. Cytochrome oxidase C subunit 1 (J01435), Cytochrome oxidase C subunit IV (X54802, M37831, AA982407), NADH dehydrogenase subunit 2 (NC_{—}001665), 12S rRNA (J01438) and F1-ATPase subunit alpha (X56133); and other genes, including neurogranin (Ng/RC3; U22062), bone morphogenetic protein 2 (Z25868), glucose-regulated protein 78 (GRP78; M19645), brain-derived neurotrophic factor (BDNF; M61178), interleukin-1β (IL-1β; D21835), dendrin (Y09000), and Ca⁺⁺/calmodulin-dependent protein kinase II (α-subunit) (J02942). Additionally, as described in Chemelli et al., Cell 98:437-451, orexin and its receptor also regulate sleep and wake in mice. Furthermore, as described in Cortelli et al, J. Sleep Res. 8(S):23-29, prior protein gene (PRNP) is associated with the vigilance disorder fatal familial insomnia.

Other rat genes, previously undisclosed as vigilance-modulated genes, identified by differential display analysis performed according to the methods described in Cirelli et al., Mol. Brain Res. 56, 293 (1998), include Cytochrome P450 (Cyp4F5) (U39206, U39207), AA117313, aryl sulfotransferase IV (X68640; S42994), human breast tumor autoantigen homolog (LM04; U24576), an apparent KIAA313 homolog (contains SEQ ID NO:15; similar to human gene AB002311), and membrane protein E25 (AF038953). Additional rat genes that are upregulated during wake contain the nucleotide sequences designated SEQ ID NOS:14 and 16-27. Other rat genes that are upregulated during sleep contain the nucleotide sequences designated SEQ ID NOS:8-13. Therefore, invertebrate homologs of each of these genes are considered to be vigilance-modulated genes.

Those skilled in the art can determine the extent of identity or similarity between two genes needed to establish that an invertebrate sequence is the homolog of a mammalian vigilance-modulated gene. Generally, homologous genes will encode polypeptides having at least about 25% identity, such as at least about 30%, 40%, 50%, 75% or greater identity across the entire sequence, or a functional domain thereof. Methods for cloning homologs from any invertebrate species, using PCR or library screening, are well known in the art, and are described, for example, in standard molecular biology manuals such as Sambrook et al., Molecular Cloning: A Laboratory Manual, Cold Spring Harbor Laboratory, New York (1992) and in Ausubel et al., Current Protocols in Molecular Biology, John Wiley and Sons, Baltimore, Md. (1998).

Another example of a molecular vigilance property that can be evaluated in invertebrates is function of one or more vigilance-altering genes. As used herein, the term “vigilance-altering gene” refers to a gene whose expression level can, but does not need to, vary with vigilance state, but whose function influences or is required for inducing or maintaining a vigilance level or a vigilance property. Exemplary functions of a vigilance-altering gene that can be evaluated include transcriptional or translational regulatory activity, and phosphorylation, dephosphorylation, glycosylation or other post-translational modification.

Vigilance-modulated genes and vigilance-altering genes can be identified, or their roles confirmed, by a variety of methods, including genetic methods. For example, animals can be generated or identified with mutations at selected or random loci, and their vigilance properties evaluated in order to determine whether vigilance-modulated or vigilance-altering genes map to these loci. For example, as described in Example IV, below, the gene for arylalkylamine N-acetyl transferase (also known as dopamine acetyltransferase, or Dat; GenBank Accession No. Y07964)) is both a vigilance-modulated gene and a vigilance-altering gene in invertebrates. Drosophila homozygous for a naturally-occurring hypomorphic allele of this gene, Dat^{lo}, exhibit a sleep rebound following sleep deprivation that is much greater than in wild-type flies, indicating that the Dat gene functions in the homeostatic regulation of sleep. Drosophila hemizygous for the Dat^{lo} mutation, generated by crossing homozygotes with Drosophila deficient at the Dat locus (Df), exhibit an even more severe sleep rebound effect. Other vigilance modulated genes and vigilance-altering genes can be identified, or their roles confirmed, by similar methods.

As described in Example IV, below, Dopa decarboxylase (Ddc) (GenBank Accession Nos. X04661, M24111, X16802; human:M88700) is a further example of a vigilance-altering gene whose function affects homeostatic regulation of sleep. More specifically, the amount of Ddc enzymatic activity in the invertebrate is directly correlated with the amount of sleep rebound exhibited by the animal following sleep deprivation, with animals severely mutant at the Ddc locus exhibiting less rebound than more mildly affected flies, and mildly affected flies exhibiting less rebound than wild-type flies.

Genetic methods of identifying new vigilance-modulated or vigilance-altering genes that are applicable to a variety of invertebrates are known in the art. For example, the invertebrate can be mutagenized using chemicals, radiation or insertions (e.g. transposons, such as P element mutagenesis), appropriate crosses performed, and the progeny screened for phenotypic differences in vigilance properties compared with normal controls. The gene can then be identified by a variety of methods including, for example, linkage analysis or rescue of the gene targeted by the inserted element. Genetic methods of identifying genes are described for Drosophila, for example, in Greenspan, Fly Pushing: The Theory and Practice of Drosophila Genetics, Cold Spring Harbor Laboratory Press (1997).

There is a distinction between genes that are modulated by vigilance state and genes that are modulated by circadian rhythms. Thus, a gene that is modulated by vigilance state will have a particular expression level during a normal wake period that is similar to the expression level following sleep deprivation, and a different expression level during a normal sleep period. In contrast, a gene that is modulated by circadian rhythms will have a particular expression level during the light period, and a different expression level during the dark period, independent of the vigilance state of the animal. As shown in Example IV, below, D-fos is an example of a gene whose expression is modulated by circadian rhythm rather than by vigilance state.

Assays to evaluate expression of vigilance-modulated genes can involve sacrificing the animal at the appropriate time, such as during a normal wake period, during a normal sleep period or following sleep deprivation, homogenizing the entire animal, or a portion containing the brain or sensory organs, and extracting either mRNA or proteins therefrom. Alternatively, such assays can be performed in biopsied tissue from the invertebrate.

A variety of assays well known in the art can be used to evaluate expression of particular vigilance-modulated genes, including the genes described above. Assays that detect mRNA expression generally involve hybridization of a detectable agent, such as a complementary primer or probe, to the nucleic acid molecule. Such assays include, for example, Northern or dot blot analysis, primer extension, RNase protection assays, reverse-transcription PCR, competitive PCR, real-time quantitative PCR (TaqMan PCR), and nucleic acid array analysis.

Additionally, constructs containing the promoter of a vigilance-modulated gene and a reporter gene (e.g. β-galactosidase, green fluorescent protein, luciferase) can be made by known methods, and used to generate transgenic invertebrates. In such transgenic invertebrates, expression of the reporter gene is a marker for expression of the vigilance-modulated gene.

Assays that detect protein expression can also be used to evaluate expression of particular vigilance-modulated genes. Such assays generally involve binding of a detectable agent, such as an antibody or selective binding agent, to the polypeptide in a sample of cells or tissue from the animal. Protein assays include, for example, immunohistochemistry, immunofluorescence, ELISA assays, immunoprecipitation, and immunoblot analysis.

Those skilled in the art will appreciate that the methods of the invention can be practiced in the absence of knowledge of the sequence or function of the vigilance-modulated genes whose expression is evaluated. Expression of vigilance-modulated genes can thus be evaluated using assays that examine overall patterns of gene expression characteristic of vigilance state. It will be understood that as these vigilance-modulated genes are identified or sequenced, specific probes, primers, antibodies and other binding agents can be used to evaluate their expression more specifically using any of the above detection methods.

One assay to examine patterns of expression of vigilance-modulated genes, that does not require prior knowledge of their sequence, is mRNA differential display, which is described, for example, in Cirelli et al., Mol. Brain Res. 56:293 (1998) and exemplified in invertebrates in Example IV, below. In such a method, RNA from the animal is reverse-transcribed and amplified by PCR using a particular combination of arbitrary primers. A detectable label, such as an enzyme, biotin, fluorescent dye or a radiolabel, is incorporated into the amplification products. The labeled products are then separated by size, such as on acrylamide gels, and detected by any method appropriate for detecting the label, including autoradiography, phosphoimaging or the like.

Such a method allows concurrent examination of expression of thousands of RNA species, the vast majority of which are expected not to be modulated by vigilance state. However, as described in Example IV, below, there will be a characteristic, reproducible banding pattern associated with vigilance state. It can be readily determined whether a particular candidate compound alters this pattern of gene expression, such as by increasing or decreasing the intensity of vigilance-modulated bands.

A further assay to examine patterns of expression of vigilance-modulated genes is array analysis, in which nucleic acids representative of all or a portion of the genome of the invertebrate, or representative of all or a portion of expressed genes of the invertebrate, are attached to a solid support, such as a filter, glass slide, chip or culture plate. Detectably labeled probes, such as cDNA probes, are then prepared from mRNA of an animal, and hybridized to the array to generate a characteristic, reproducible pattern of spots associated with vigilance state. It can be readily determined whether a particular candidate compound alters this pattern of gene expression, such as by increasing or decreasing the intensity of vigilance-modulated spots.

Following identification of patterns of vigilance-modulated gene expression, those skilled in the art can clone the genes, if desired, using standard molecular biology approaches. For example, a vigilance-modulated band identified by differential display can be eluted from a gel and sequenced, or used to probe a library to identify the corresponding cDNA or genomic DNA. Likewise, a vigilance-modulated gene from an array can be identified based on its known position on the array, or cloned by PCR or by probing a library.

If desired, any of the expression and activity assays described above can be used in combination, either sequentially or simultaneously. Such assays can also be partially or completely automated, using methods known in the art.

Given the teachings described herein that behavioral vigilance properties are closely correlated with molecular vigilance properties, and that behavioral and molecular properties are highly conserved across disparate species, for example, mammals and flies, it is understood that the invention can be practiced using any invertebrate that exhibits at least one behavioral or one molecular vigilance property that is susceptible to evaluation or measurement.

As disclosed herein, Drosophila melanogaster is an example of an invertebrate that exhibits a variety of vigilance properties that can be evaluated, including homeostatically regulated activity, arousal threshold, latency to sleep, and expression of vigilance-modulated genes. Those skilled in the art understand that other Drosophila species are also likely to exhibit similar vigilance properties, including D. simulans, D. virilis, D. pseudoobscura D. funebris, D. immigrans, D. repleta, D. affinis, D. saltans, D. sulphurigaster albostrigata and D. nasuta albomicans. Likewise, other flies, including, sand flies, mayflies, blowflies, flesh flies, face flies, houseflies, screw worm-flies, stable flies, mosquitos, northern cattle grub, and the like will also exhibit vigilance properties.

Furthermore, insects other than flies can also exhibit behavioral and molecular vigilance properties. For example, species of cockroach exhibit rest rebound following rest deprivation, as well as a higher arousal threshold correlated with rest (Tobler et al., Sleep Res. 1:231-239 (1992)). Thus, the invention can also be practiced with insects such as cockroaches, honeybees, wasps, termites, grasshoppers, moths, butterflies, fleas, lice, boll weevils and beetles.

Arthropods other than insects also can exhibit behavioral and molecular vigilance properties. For example, scorpions exhibit rest rebound following rest deprivation, as well as a characteristic arousal threshold and heart rate associated with rest (Tobler et al., J. Comp. Physiol. 163:227-235 (1988)). Thus, the invention can also be practiced using arthropods such as scorpions, spiders, mites, crustaceans, centipedes and millipedes.

Due to the high degree of genetic similarity across invertebrate species, invertebrates other than arthropods, such as flatworms, nematodes (e.g. C. elegans), mollusks (e.g. Aplysia or Hermissenda), echinoderms and annelids will exhibit behavioral and molecular properties correlated with vigilance state, and can be used in the methods of the invention.

Those skilled in the art can determine, using the assays described herein, whether a particular invertebrate exhibits behavioral or molecular properties correlated with vigilance state and, therefore, would be applicable for use in the methods of the invention. The choice of invertebrate will also depend on additional factors, for example, such as the availability of the animals, the normal activity levels of the animals, the availability of molecular probes for vigilance-modulated genes, the number of animals and compounds one intends to screen, the ease and cost of maintaining the animals in a laboratory setting, the method of contacting and type of compounds being tested, and the particular property being evaluated. Those skilled in the art can evaluate these factors in determining an appropriate invertebrate to use in the screening methods.

For example, if it is desired to evaluate molecular properties in the methods of the invention, an invertebrate that is genetically well-characterized, such that homologs of vigilance-modulate genes are known or can be readily determined, may be preferred. Thus, appropriate invertebrates in which to evaluate molecular properties of vigilance can include, for example, Drosophila, and C. elegans. If it desired to evaluate behavioral properties in the methods of the invention, an invertebrate that exhibits one or more behavioral properties now known to be consistent with sleep, such as fruit flies, cockroaches, honeybees, wasps, moths, mosquitos, scorpions, may be preferred.

As disclosed herein, invertebrate sleep exhibits an age-dependence similar to mammalian sleep. Therefore, it may be desirable to practice the methods of the invention using invertebrates of different ages so as to identify compounds that alter vigilance in the very young or very old. Such compounds can be tailored for use in pediatric or geriatric patients.

As also disclosed herein, invertebrate sleep patterns differ between females and males. Therefore, it may be desirable to practice the methods of the invention using invertebrates of both genders separately to identify compounds appropriate for use in females, males, or both females and males.

If desired, invertebrates that contain mutations of varying degrees of severity in vigilance-altering genes can be used in the screening methods described herein, and compounds identified that correct these defects. In such screens, a vigilance property is evaluated in mutant invertebrates and in normal invertebrates. A compound that alters the vigilance property in the mutant invertebrate to a level or amount more similar to the property in the normal animal can thus be identified. For example, a screen can be conducted in a Drosophila that is mutant at the Dat locus or the Ddc locus, both of which, as shown in Example IV, alter, in different directions, the amount of sleep rebound exhibited by the animal following sleep deprivation. Accordingly, a compound that alters homeostatic regulation of sleep can be identified as a compound that restores more normal sleep rebound in a Dat or a Ddc mutant animal. Animals mutant in other vigilance-modulated or vigilance-altering genes can similarly be identified or generated, and used to identify compounds that affect a particular function implicated in vigilance (e.g. neurotransmitter synthesis or degradation), or a particular property of vigilance, including a homeostatically regulated property of vigilance.

The methods of the invention are practiced by contacting an invertebrate with a candidate compound, and evaluating a vigilance property. Appropriate invertebrates, candidate compounds and vigilance properties to evaluate for various applications of the method have been described above. As used herein, the term “contacting” refers to any method of administering a candidate compound to an invertebrate such that the compound, or a metabolite thereof, is introduced into the invertebrate in an effective amount so as to act on its nervous system.

Exemplary methods of contacting an invertebrate with a candidate compound include feeding the compound to the animal, topical administration of the compound, administration by aerosol spray, immersion of the animal in a solution containing the compound, and injection of the compound. An appropriate method of contacting an invertebrate with a compound can be determined by those skilled in the art and will depend, for example, on the type and developmental stage of the invertebrate, whether the invertebrate is sleeping or awake at the time of contacting, the number of animals being assayed, and the chemical and biological properties of the compound (e.g. solubility, digestibility, bioavailability, stability and toxicity). For example, as shown in Example IV below, Drosophila melanogaster can be contacted with stimulants or hypnotics by dissolving the drugs in fly food and providing the food to the flies.

A “candidate compound” used to contact the invertebrate can be any molecule that potentially alters vigilance. A candidate compound can be a naturally occurring macromolecule, such as a peptide, nucleic acid, carbohydrate, lipid, or any combination thereof, or a partially or completely synthetic derivative, analog or mimetic of such a macromolecule. A candidate compound can also be a small organic or inorganic molecule, either naturally occurring, or prepared partly or completely by synthetic methods. If desired, a candidate compound can be combined with, or dissolved in, an agent that facilitates uptake of the compound by the invertebrate, such as an organic solvent (e.g. DMSO, ethanol), aqueous solvent (e.g. water or a buffer), or food.

A candidate compound can be tested at a single dose, or at a range of doses. It is expected that the effects on properties correlated with vigilance will be dose dependent, as demonstrated with caffeine and hydroxyzine in Example III, below. Appropriate concentrations of candidate compound to test in the methods of the invention can be determined by those skilled in the art, and will depend on the chemical and biological properties of the compound and the method of contacting. Exemplary concentration ranges to test include from about 10 μg/ml to about 500 mg/ml, such as from about 100 μg/ml to 250 mg/ml, including from about 1 mg/ml to 200 mg/ml.

The number of different compounds to screen in the methods of the invention can be determined by those skilled in the art depending on the application of the method. For example, a smaller number of candidate compounds would generally be used if the type of compound that is likely to alter vigilance is known or can be predicted, such as when derivatives of a lead compound are being tested. However, when the type of compound that is likely to alter vigilance is unknown, it is generally understood that the larger the number of candidate compounds screened, the greater the likelihood of identifying a compound that alters vigilance. Therefore, the methods of the invention can employ screening individual compounds separately or populations of compounds including small populations and large or diverse populations, to identify a compound that alters vigilance.

The appropriate time and duration to administer the compound can be determined by those skilled in the art depending on the application of the method. For example, it may be desirable to administer a compound at the beginning or end of the normal wake or sleep period, continuously throughout a normal wake or sleep period, or prior to, during, or after sleep deprivation, depending on the vigilance property being evaluated and the desired effect of the compound. As exemplified in Example III, below, compounds that either increase or decrease vigilance can be administered in the last hour of the normal wake period, and their effect on activity during the next sleep period or on latency to sleep can be readily observed.

Methods for producing libraries of candidate compounds to use in the methods of the invention, including chemical or biological molecules such as simple or complex organic molecules, metal-containing compounds, carbohydrates, peptides, proteins, peptidomimetics, glycoproteins, lipoproteins, nucleic acids, antibodies, and the like, are well known in the art. Libraries containing large numbers of natural and synthetic compounds also can be obtained from commercial sources.

Following contacting the invertebrate with the candidate compound, any of the vigilance properties described above can be evaluated, and a determination made as to whether the compound alters, such as increases or decreases, the vigilance property compared to a baseline or established value for the property in an untreated control. Such a compound will similarly alter vigilance in mammals. However, it will be understood that the efficacy and safety of the compound in laboratory mammals can be further evaluated before administering the compound to humans or veterinary animals. For example, the compound can be tested for its maximal efficacy and any potential side-effects using several different invertebrates or laboratory mammals, across a range of doses, in a range of formulations, and at various times during the normal sleep and wake periods.

Additionally, a compound that alters vigilance can be tested for its effects on one or more additional vigilance properties in order to determine its most effective application in therapy. For example, it may be desirable to determine whether a compound that increases vigilance does so without significantly altering latency to sleep when the effect of the compound wears off. Such a compound would be an improvement over many of the currently known vigilance-enhancing drugs that cause a characteristic “crash” afterwards. It may also be desirable to determine whether the compound that alters vigilance does so without a compensatory sleep rebound effect.

Therefore, once a compound is identified that alters a desirable vigilance property, the methods of the invention can be used to determine other vigilance characteristics of the compound. Such other characteristics can be assessed either simultaneously with the initial screen, or alternatively they can be assessed in or more separate screens to identify or characterize other vigilance properties of the compound. For example, a vigilance altering compound identified that promotes sleep can be further assessed to determine whether that compound additionally reduces arousal threshold to normal sleep levels, while preserving the ability of the animal to be wakened normally, and with subsequent normal wake-like behaviors. Such a compound would be an improvement over many of the currently available sleep-inducing drugs, which may not promote truly restorative sleep or normal function on awakening. Similarly, a vigilance altering compound identified that promotes wakefulness can be further assessed, as described above, to determine whether that compound additionally reduces the rate or extent of the wake-sleep transition, or “crash,” following the vigilance enhancing effects of the compound.

The methods of the invention are therefore applicable for screening and identifying compounds that exhibit preferred vigilance altering effects as well as for identifying compounds that exhibit a combination of preferred vigilance altering effects to yield optimal vigilance altering compounds. Such optimal vigilance altering compounds can be identified which combine preferred effects on vigilance levels together with maintaining some or all hemostatic regulatory properties of vigilance.

As used herein, “homestatic regulatory properties of vigilance” or “homeostatic regulatory properties” is intended to mean those vigilance properties that are compensatory changes in vigilance resulting from, or correlating with, the quantity or quality of vigilance from a previous time period. Homeostatic regulatory properties are therefore vigilance properties when viewed in light of the vigilance state of a previous period. Such properties include, for example, vigilance properties such as sleep rebound, wake period, latency to sleep, the rate of the sleep-wake transition, alertness or drowsiness when there has been a corresponding and opposite change in vigilance in the immediate, prior period, or when there has been a correlative effect in the immediate, prior period.

For the specific homeostatic regulatory property referred to as sleep rebound, prolonged or more intense sleep periods occur as a compensatory change to prior increases in vigilance periods. For the remaining homeostatic regulatory properties specifically exemplified above, such properties are, for example, compensatory changes due to correlative effects in the prior period. For example, the transition rate between wake and sleep states will be correspondingly increased or decreased depending on the amount and quality of the previous wake or sleep vigilance state. Similarly, an animal will be more alert following a more restful period and will be more drowsy following a less restful period. Such compensatory vigilance states arise from the quality and nature of vigilance state of the previous time period. Homeostatic regulatory properties of vigilance other than those described above also exist and are well known to those skilled in the art.

Preferred or optimal vigilance altering compounds can be identified using the methods of the invention which exhibit, for example, predetermined effects on the magnitude of vigilance levels or on the period and duration of the effect. For example, vigilance altering compounds can be identified that either increase or decrease vigilance levels in small or large increments or to a specified degree. Vigilance altering compounds similarly can be identified that increase or decrease vigilance levels to a maximum amount allowable without affecting other vital or relevant physiological processes. Preferred or optimal compounds also can be selected that modulate the duration of the vigilance altering effect for a predetermined period, including maximal durations, without adversely affecting other vital or relevant physiological processes.

Compounds exhibiting one or more combinations of the above effects can similarly be identified using the methods of the invention. A specific example of one such preferred or optimal combination is a compound that alters vigilance, either by increasing or decreasing vigilance, to its maximal extent, but for a short and specified time. Another example is a compound that results in small alterations in vigilance levels but exhibits a relatively prolonged, and predetermined duration of the effect. Vigilance altering compounds exhibiting other combinations of preferred or optimal vigilance effects can similarly be selected using the methods of the invention, given the teachings and descriptions herein.

Additionally, preferred or optimal vigilance altering compounds can be identified using the methods of the invention which modulate, for example, one or more homeostatic regulatory properties of vigilance following a prior perturbation in vigilance levels or periods. For example, vigilance altering compounds can be identified that modulate the sleep rebound, wake period, latency to sleep, the rate of the sleep-wake transition, alertness or drowsiness. Vigilance altering compounds can be identified, for example, that increase or decrease the period or amount of sleep rebound following prolonged periods of increased vigilance. Similarly, vigilance altering compounds can be identified, for example, that increase or decrease the period or amount of wake period as well as the level of vigilance following prolonged periods of sleep. Such compounds can be preferred because they increase the animal's alertness and therefore decrease lethargic periods during the wake state. Finally, vigilance altering compounds can be identified that, for example, decrease the rate of the wake-to-sleep transition so as to prevent a crash following prolonged waking periods as well as increase the rate of the sleep-to-wake transitions so as to achieve normal levels of vigilance following prolonged or induced periods of sleep.

Vigilance altering compounds exhibiting one or more combinations of the above modulatory effects on homeostatic regulatory properties can similarly be identified using the methods of the invention. One specific example is a compound that prevents or reduces sleep rebound to a specified extent and maintains normal vigilance levels following prolonged wake periods. Another specific example is a compound that increases the rate of the sleep-to-wake transition while also preventing lethargic periods during the wake state following prolonged or induced sleep.

Likewise, the methods of the invention are also applicable to identifying compounds that maintain or mimic, for example, one or more homeostatic regulatory properties following a prior perturbation. For example, it can be desirable to maintain or induce normal homeostatic regulatory properties following prior preturbation of vigilance levels or periods. In such instances, the methods of the invention can be used to identify compounds that cause such effects following a prior modulation of vigilance.

Finally, preferred or optimal vigilance altering compounds can be identified using the methods of the invention which exhibit combinations, including optimal combinations, of one or more preferred vigilance altering effects and modulation or maintenance of one or more homeostatic regulatory properties of vigilance. For example, vigilance altering compounds can be identified that induce specific magnitudes or durations of vigilance levels and which alter homeostatic regulatory properties following the induced changes in vigilance levels. One specific example, is a compound that maximally increases vigilance levels over prolonged periods without a subsequent sleep rebound effect. Alternatively, such a vigilance increasing compound can also result in little or no crash following the prolonged wake period. Another example is a compound that decreases vigilance, such as induces restful sleep states, for a predetermined period without a lethargic vigilance states following the sleep period. Similarly, vigilance altering compounds can be identified that induce specific magnitudes or durations of vigilance levels and which alter homeostatic regulatory properties simultaneously with the induced changes in vigilance levels. Compounds exhibiting various other combinations of vigilant altering effects and modulation, or maintenance, of homeostatic regulatory properties can similarly be identified using the method and teachings described herein.

Therefore, the invention allows the identification of compounds that alter vigilance levels and modulate or maintain homeostatic regulatory properties of vigilance. Such compounds can be identified in the initial screen, or alternatively, such compounds can be identified step-wise by first identifying compounds that alter vigilance and subsequently determining whether such identified compounds affect homeostatic regulatory properties of vigilance, such as sleep rebound and latency to sleep. Similarly, compounds can be identified either in the initial screen or in step-wise procedures that alter vigilance properties and are devoid of deleterious side-effects, such as a precipitous crash after the drug wears off or lack of restfulness following drug induced sleep. Therefore, the methods of the invention are applicable for identifying compounds that alter vigilance in mammals, as well as to identifying compounds that alter vigilance levels with concomitant homeostatic regulatory properties. Similarly, the methods of the invention are also applicable to identifying compounds that alter vigilance in mammals that are devoid of deleterious and unwanted side-effects.

Compounds identified by the methods of the invention as compounds that alter vigilance can also have an effect on neuronal plasticity, or the ability to learn and form memories. Learning is not possible during sleep in mammals, whereas learning and memory are positively associated with the level of vigilance during waking. Thus, by increasing vigilance, it is also possible to increase learning and memory. Accordingly, in one embodiment, the invertebrate is contacted with a candidate compound, a vigilance property is evaluated, and learning or memory is also evaluated.

A variety of assays are known in the art that can be used to evaluate learning and either short-term or long-term memory in invertebrates, including habituation and sensitization assays, and conditioning assays. Habituation refers to a decrease, and sensitization refers to an increase, in a behavioral response on repeated presentation of the same stimulus, and can be considered rudimentary forms of learning. Exemplary habituation assays that can be readily adapted for use in a variety of invertebrates are described, for example, for C. elegans in Rankin et al., Behav. Brain Res. 37:89-92 (1990); for Drosophila in Boynton et al., Genetics 131:655-672 (1992); and for Aplysia in Kandel et al., Cold Spring Harb. Symp. Quant. Biol. 40:465-482 (1976).

Classical (Pavlovian) conditioning is an accepted behavioral paradigm for learning and memory. In an exemplary conditioning assay, invertebrates can be exposed to two different stimuli, such as two odorants or two colors of light, one of which is associated with negative reinforcement, such as an electric shock. The animals are then removed and tested in a new apparatus, similar to the training arrangement but without reinforcement. Avoidance behavior is scored as learning, and retention time of the learned behavior is scored as memory. Exemplary conditioning assays that can be readily adapted for use in a variety of invertebrates are described, for example, for Drosophila in Quinn et al., Proc. Natl. Acad. Sci. USA 71:708-712 (1974); for cockroach in Mizunami et al., J. Comp. Neruol. 402:520-537 (1998); and for crab in Hoyle, Behav. Biol. 18:147-163 (1976).

As described previously, invertebrate sleep, exemplified by Drosophila sleep, is comparable to mammalian sleep by behavioral, physiological, developmental, molecular and genetic criteria. In particular, individual genes, and classes of genes, identified as vigilance-modulated genes in Drosophila are also vigilance-modulated in mammals (see Example IV). Other vigilance-modulated genes identified from invertebrate molecular and genetic screens thus will also likely be vigilance-modulated in mammals.

As exemplified in Example IV using mutants in the Dat gene, deliberately altering the activity or expression of vigilance-modulated genes in invertebrates is an effective method of altering a desired vigilance property. As further exemplified in Example IV using mutants in the Ddc gene, deliberately altering the activity or expression of genes that are vigilance-altering, but not necessarily vigilance-modulated, in invertebrates is also an effective method of altering a desired vigilance property. Deliberately altering the activity or expression of vigilance-modulated or vigilance-altering genes in mammals, collectively termed henceforth as “vigilance genes,” are thus expected to be similarly effective in altering desired vigilance properties.

There are numerous important diagnostic, therapeutic, and screening applications that arise from identification of novel vigilance genes, together with knowledge that modulation of expression or activity of such vigilance genes is an effective method of altering vigilance. For example, an expression or activity profile of one or many vigilance genes can be established that is a molecular fingerprint of each mammalian vigilance level, state or disorder of interest. Thus, in diagnostic applications, it can readily be determined, by comparing the vigilance gene profile of the individual to control profiles, whether that individual suffers from, or is susceptible to, a particular vigilance disorder. Likewise, the vigilance level of an individual, and the effect of medications or medical procedures on the vigilance level, can be accurately determined at the molecular level. Such determinations allow for more appropriate determination and use of therapeutics for treating vigilance disorders and for maintaining or restoring normal sleep and wake patterns.

In screening applications, identification of vigilance genes and their role in vigilance allows novel vigilance-altering compounds to be identified, lead compounds to be validated, and the molecular effects of these compounds and other known vigilance-altering compounds to be characterized, by determining the effect of these compounds on a vigilance gene profile. For example, the ability of a compound to alter a vigilance gene profile of an individual to correspond more closely to a desired vigilance level or state can be determined. Likewise, the ability of a compound, administered to an individual with a particular vigilance disorder, to alter the vigilance gene profile to correspond more closely to the profile of a normal individual can be determined. The compounds so identified, validated or characterized from such assays can be administered to normal individuals to enhance or reduce vigilance, as desired, or to individuals having a vigilance disorder to ameliorate the disorder and induce more normal sleep and wake patterns.

The invention thus provides an isolated vigilance nucleic acid molecule, containing a nucleic acid sequence selected from the group consisting of SEQ ID NOS:1-6 and 8-27, or a modification thereof. An isolated nucleic acid molecule containing a nucleotide sequence designated SEQ ID NO:15, or modification thereof, will not consist of the exact sequence of the human KIAA313 gene having GenBank Accession No. AB002311. An isolated nucleic acid molecule containing a nucleotide sequence designated SEQ ID NO:1, or modification thereof, will not consist of the exact sequence of the Drosophila P1 clone having GenBank Accession No. AC005554.

In one embodiment, an isolated vigilance nucleic acid molecule of the invention contains a nucleic acid sequence selected from the group consisting of SEQ ID NOS:1-6 and 8-27. An isolated nucleic acid molecule containing a nucleotide sequence designated SEQ ID NO:1 will not consist of the exact sequence of the Drosophila P1 clone having GenBank Accession No. AC005554. In another embodiment, an isolated vigilance nucleic acid molecule of the invention consists of a nucleic acid sequence selected from the group consisting of SEQ ID NOS:1-6 and 8-27.

The isolated vigilance nucleic acid molecules of the invention contain sequences from novel vigilance-modulated genes identified from mRNA differential display analysis performed in Drosophila melanogaster (SEQ ID NOS:1-6), or in rat (SEQ ID NOS:8-27). SEQ ID NOS:2, 3 and 8-13 correspond to genes that are upregulated during sleep. SEQ ID NOS:4, 5, 6 and 14-27 correspond to genes that are upregulated during wake.

The isolated vigilance nucleic acid molecules of the invention hybridize to mammalian vigilance genes, and thus can be used in the diagnostic and screening methods described below. Additionally, the isolated vigilance nucleic acid molecules of the invention can be administered in gene therapy methods, including antisense and ribozyme methods, to increase or decrease expression of encoded vigilance polypeptides. The isolated vigilance nucleic acid molecules of the invention can also be used as probes or primers to identify larger vigilance cDNAs or genomic DNA, or to identify homologs of the vigilance nucleic acid molecules in other species. The isolated vigilance nucleic acid molecules can further be expressed to produce vigilance polypeptides for use in producing antibodies or for rationally designing inhibitory or stimulatory compounds. Other uses for the isolated vigilance nucleic acid molecules of the invention can be determined by those skilled in the art.

As used herein, the term “nucleic acid molecule” refers to both deoxyribonucleic acid (DNA) and ribonucleic acid (RNA) molecules, and can optionally include one or more non-native nucleotides, having, for example, modifications to the base, the sugar, or the phosphate portion, or having a modified phosphodiester linkage. The term nucleic acid molecule includes both single-stranded and double-stranded nucleic acids, representing the sense strand, the anti-sense strand, or both, and includes linear, circular or branched molecules. Exemplary nucleic acid molecules include genomic DNA, cDNA, mRNA and oligonucleotides, corresponding to either the coding or non-coding portion of the molecule, and optionally containing sequences required for expression. A nucleic acid molecule of the invention, if desired, can additionally contain a detectable moiety, such as a radiolabel, a fluorochrome, a ferromagnetic substance, a luminescent tag or a detectable agent such as biotin.

The term “isolated” in reference to a vigilance nucleic acid molecule is intended to mean that the molecule is substantially removed or separated from components with which it is naturally associated, or otherwise modified by a human hand, thereby excluding vigilance nucleic acid molecules as they exist in nature. An isolated nucleic acid molecule of the invention can be in solution or suspension, or immobilized on a filter, glass slide, chip, culture plate or other solid support. The degree of purification of the nucleic acid molecule, and its physical form, can be determined by those skilled in the art depending on the intended use of the molecule.

The term “comprising” or “containing” in reference to a vigilance nucleic acid molecule of the invention, is intended to mean that the nucleic acid molecule can contain additional nucleotide sequences at either the 5′ or 3′ end of the recited sequence, or branching from an internal position within the recited sequence. The additional nucleotide sequences can, if desired, correspond to sequences that naturally occur within the vigilance gene, including intron or exon sequences, promoter sequences, coding sequence, or untranslated regions. Alternatively, the additional nucleotide sequence can correspond to linkers or restriction sites useful in cloning applications; to other regulatory elements such as promoters and polyadenylation sequences that can be useful in gene expression; to epitope tags or fusion proteins useful in protein purification; or the like. Those skilled in the art can determine appropriate sequences flanking the recited nucleotide sequences for a particular application of the method.

The term “modification,” in reference to a vigilance nucleic acid molecule of the invention, is intended to mean a nucleic acid molecule that contains one or several nucleotide additions, deletions or substitutions with respect to a reference sequence, yet retains at least one function specific to the reference sequence. The appropriate function to be retained will depend on the desired use of the nucleic acid molecule. For example, where it is desired to express a vigilance polypeptide, a “modification” can encode substantially the same polypeptide as the reference vigilance nucleic acid molecule, such that the encoded polypeptide has substantially the same immunogenicity, antigenicity, enzymatic activity, binding activity, or other biological property, including vigilance-altering therapeutic activity, as the polypeptide encoded by the reference vigilance nucleic acid molecule.

Where it is desired to use a vigilance nucleic acid molecule in the diagnostic and screening assays described herein, a “modification” of a vigilance nucleic acid molecule can be a molecule that retains the ability to hybridize to the recited sequence under moderately stringent conditions, or under highly stringent conditions. The term “moderately stringent conditions,” is intended to refer to hybridization conditions equivalent to hybridization of filter-bound nucleic acid in 50% formamide, 5×Denhart's solution, 5×SSPE, 0.2% SDS at 42° C., followed by washing in 0.2×SSPE, 0.2% SDS, at 50°. In contrast, “highly stringent conditions” are conditions equivalent to hybridization of filter-bound nucleic acid in 50% formamide, 5×Denhart's solution, 5×SSPE, 0.2% SDS at 42° C., followed by washing in 0.2×SSPE, 0.2% SDS, at 65°. Other suitable moderately stringent and highly stringent hybridization buffers and conditions are well known to those of skill in the art and are described, for example, in Sambrook et al., Molecular Cloning: A Laboratory Manual, Cold Spring Harbor Laboratory, New York (1992) and in Ausubel et al., Current Protocols in Molecular Biology, John Wiley and Sons, Baltimore, Md. (1998).

Thus, a modification of a vigilance nucleic acid molecule can be a sequence that corresponds to a homolog of the vigilance gene in another animal species, including other Drosophila species, other flies, other arthropods, other invertebrates, as well as other mammalian species, such as human, primates, rat, mouse, rabbit, bovine, porcine, canine or feline. The sequences of corresponding vigilance genes of desired species can be determined by methods well known in the art, such as by PCR or by screening genomic, cDNA or expression libraries derived from that species.

A modification of a vigilance nucleic acid molecule can also include substitutions that do not change the encoded amino acid sequence due to the degeneracy of the genetic code. Such modifications can correspond to variations that are made deliberately, or which occur as mutations during nucleic acid replication. Additionally, a modification of a vigilance nucleic acid molecule can correspond to a splice variant form of the recited sequence.

In general, a modification of a vigilance nucleic acid molecule of the invention that retains at least one function specific to the reference sequence will have greater than about 60% identity, such as greater than about 70% identity, including greater than about 80%, 90%, 95%, 97% or 99% identity, to the reference sequence over the length of the two sequences being compared. Identity of any two nucleic acid sequences can be determined by those skilled in the art based, for example, on a BLAST 2.0 computer alignment, using default parameters. BLAST 2.0 alignments can be performed at http://www.ncbi.nlm.nih.gov/gorf/bl2.html, as described by Tatiana et al., FEMS Microbiol Lett. 174:247-250 (1999).

The invention also provides isolated oligonucleotides containing at least 15 contiguous nucleotides of a nucleotide sequence referenced as SEQ ID NOS:1-6 and 8-27, or the antisense strand thereof. The isolated oligonucleotides of the invention are able to hybridize to vigilance nucleic acid molecules under moderately stringent hybridization conditions and thus can be advantageously used, for example, as probes to detect vigilance gene DNA or RNA in a sample; as sequencing or PCR primers; as antisense reagents to administer to an individual to block translation of vigilance RNA in cells; or in other applications known to those skilled in the art in which hybridization to a vigilance nucleic acid molecule is desirable.

As used herein, the term “oligonucleotide” refers to a nucleic acid molecule that includes at least 15 contiguous nucleotides from the reference nucleotide sequence, can include at least 16, 17, 18, 19, 20 or at least 25 contiguous nucleotides, and often includes at least 30, 40, 50, 60, 70, 80, 90, 100, 125, 150, 175, 200 or more contiguous nucleotides from the reference nucleotide sequence.

If desired, the oligonucleotide containing at least 15 contiguous nucleotides of a nucleotide sequence referenced as SEQ ID NOS:1-6 and 8-27 can further be capable of specifically hybridizing with the reference nucleic acid molecule. As used herein, the term “specifically hybridize” refers to the ability of a nucleic acid molecule to hybridize, under moderately stringent conditions as described above, to the reference nucleic acid molecule, without substantial hybridization under the same conditions with nucleic acid molecules that are not the reference nucleic acid molecules. Those skilled in the art can readily determine whether an oligonucleotide of the invention both hybridizes to the recited nucleic acid sequence under moderately stringent conditions, and also is able to specifically hybridize to the sequence, by performing a hybridization assay in the presence of other nucleic acid molecules, such as total cellular nucleic acid molecules, and detecting the presence or absence of hybridization to the other nucleic acid molecules.

Depending on the intended use of the oligonucleotides of the invention, those skilled in the art can determine whether it is necessary to use an oligonucleotide that hybridizes to the recited vigilance nucleic acid molecule and that also specifically hybridizes to the recited vigilance nucleic acid molecules. For example, when there are a large number of potential contaminating nucleic acid molecules in the sample, it may be desirable to use an oligonucleotide that specifically hybridizes to the recited vigilance nucleic acid molecule. However, when background hybridization is not considered detrimental, when there are few contaminating molecules, or when the oligonucleotide is being used in conjunction with a second molecule, such as a second primer, an oligonucleotide of the invention can be used that does not specifically hybridize to the recited nucleic acid sequence.

In one embodiment, the invention provides a primer pair for detecting vigilance nucleic acid molecules. The primer pair contains two isolated oligonucleotides, each containing at least 15 contiguous nucleotides of one of the nucleotide sequences referenced as SEQ ID NOS:1-6 and 8-27, with one sequence representing the sense strand, and one sequence representing the anti-sense strand. The primer pair can be used, for example, to amplify vigilance nucleic acid molecules by RT-PCR or PCR.

The isolated vigilance nucleic acid molecules and oligonucleotides of the invention can be produced or isolated by methods known in the art. The method chosen will depend, for example, on the type of nucleic acid molecule one intends to isolate. Those skilled in the art, based on knowledge of the nucleotide sequences disclosed herein, can readily isolate the vigilance nucleic acid molecules of the invention as genomic DNA, or desired introns, exons or regulatory sequences therefrom; as full-length cDNA or desired fragments therefrom; or as full-length mRNA or desired fragments therefrom, by methods known in the art.

One useful method for producing an isolated vigilance nucleic acid molecule of the invention involves amplification of the nucleic acid molecule using the polymerase chain reaction (PCR) and vigilance nucleic acid-specific oligonucleotide primers and, optionally, purification of the resulting product by gel electrophoresis. Either PCR or reverse-transcription PCR (RT-PCR) can be used to produce a vigilance nucleic acid molecule having any desired nucleotide boundaries. Desired modifications to the nucleic acid sequence can also be introduced by choosing an appropriate primer with one or more additions, deletions or substitutions. Such nucleic acid molecules can be amplified exponentially starting from as little as a single gene or mRNA copy, from any cell, tissue or species of interest.

A further method of producing an isolated vigilance nucleic acid molecule of the invention is by screening a library, such as a genomic library, cDNA library or expression library, with a detectable agent. Such libraries are commercially available or can be produced from any desired tissue, cell, or species of interest using methods known in the art. For example, a cDNA or genomic library can be screened by hybridization with a detectably labeled nucleic acid molecule having a nucleotide sequence disclosed herein. Additionally, an expression library can be screened with an antibody raised against a polypeptide encoded by a vigilance nucleic acid disclosed herein. The library clones containing vigilance molecules of the invention can be isolated from other clones by methods known in the art and, if desired, fragments therefrom can be isolated by restriction enzyme digestion and gel electrophoresis.

Furthermore, isolated vigilance nucleic acid molecules and oligonucleotides of the invention can be produced by synthetic means. For example, a single strand of a nucleic acid molecule can be chemically synthesized in one piece, or in several pieces, by automated synthesis methods known in the art. The complementary strand can likewise be synthesized in one or more pieces, and a double-stranded molecule made by annealing the complementary strands. Direct synthesis is particularly advantageous for producing relatively short molecules, such as oligonucleotide probes and primers, and nucleic acid molecules containing modified nucleotides or linkages.

In one embodiment, the isolated vigilance nucleic acid molecules or oligonucleotides of the invention are attached to a solid support, such as a chip, filter, glass slide or culture plate, by either covalent or non-covalent methods. Methods of attaching nucleic acid molecules to a solid support, and the uses of nucleic acids in this format in a variety of assays, including manual and automated hybridization assays, are well known in the art. A solid support format is particularly appropriate for automated diagnostic or screening methods, where simultaneous hybridization to a large number of vigilance genes is desired, or when a large number of samples are being handled.

In another embodiment, the invention provides kits containing two or more isolated vigilance nucleic acid molecules or oligonucleotides. At least one vigilance nucleic acid molecule contains a nucleotide sequence selected from the group consisting of SEQ ID NOS:1-6 and 8-27 or modification thereof. An exemplary kit is a solid support containing an array of isolated vigilance nucleic acid molecules or oligonucleotides of the invention, including, for example, at least 3, 5, 10, 20, 30, 40, 50, 75, 100 or more isolated vigilance nucleic acid molecules or oligonucleotides.

A further exemplary kit contains one or more PCR primer pairs, or two or more hybridization probes, which optionally can be labeled with a detectable moiety for detection of vigilance nucleic acid molecules. The kits of the invention can additionally contain instructions for use of the molecules for diagnostic purposes in a clinical setting, or for drug screening purposes in a laboratory setting.

If desired, the kits containing two or more isolated vigilance nucleic acid molecules or oligonucleotides can contain nucleic acid molecules corresponding to genes that are upregulated during sleep, during wake, or any combination of these genes. Additionally, the kits containing two or more isolated vigilance nucleic acid molecules or oligonucleotides can contain nucleic acid molecules corresponding to sequences identified from Drosophila screens, from rat screens, from screens in other animals, or any combination thereof.

The invention also provides a vector containing an isolated vigilance nucleic acid molecule. The vectors of the invention are useful for subcloning and amplifying an isolated vigilance nucleic acid molecule, for recombinantly expressing a vigilance polypeptide, and in gene therapy applications, described further below. A vector of the invention can include a variety of elements useful for cloning and/or expression of vigilance nucleic acid molecules, such as enhancer sequences and promoter sequences from a viral, bacterial or mammalian gene, which provide for constitutive, inducible or cell-specific RNA transcription; transcription termination and RNA processing signals, including polyadenylation signals, which provide for stability of a transcribed mRNA sequence; an origin of replication, which allows for proper episomal replication; selectable marker genes, such as a neomycin or hygromycin resistance gene, useful for selecting stable or transient transfectants in mammalian cells, or an ampicillan resistance gene, useful for selecting transformants in prokaryotic cells; and versatile multiple cloning sites for inserting nucleic acid molecules of interest.

A variety of cloning and expression vectors are commercially available, and include, for example, viral vectors such as a bacteriophage, baculovirus, adenovirus, adeno-associated virus, herpes simplex virus and retrovirus; cosmids or plasmids; bacterial artificial chromosome vectors (BACs) and yeast artificial chromosome vectors (YACs). Such vectors and their uses are well known in the art.

The invention also provides host cells that contain a vector containing a vigilance nucleic acid molecule of the invention. Exemplary host cells include mammalian primary cells; established mammalian cell lines, such as COS, CHO, HeLa, NIH3T3, HEK 293-T and PC12 cells; amphibian cells, such as Xenopus embryos and oocytes; and other vertebrate cells. Exemplary host cells also include insect cells (e.g. Drosophila), yeast cells (e.g. S. cerevisiae, S. pombe, or Pichia pastoris) and prokaryotic cells (e.g. E. coli). Methods of introducing a vector of the invention into such host cells are well known in the art.

The methods of isolating, cloning and expressing nucleic acid molecules of the invention referred to herein are routine in the art and are described in detail, for example, in Sambrook et al., Molecular Cloning: A Laboratory Manual, Cold Spring Harbor Laboratory, New York (1992) and in Ansubel et al., Current Protocols in Molecular Biology, John Wiley and Sons, Baltimore, Md. (1998), which are incorporated herein by reference.

The invention further provides transgenic non-human animals that are capable of expressing wild-type vigilance nucleic acids, dominant-negative vigilance nucleic acids, antisense vigilance nucleic acids, or ribozymes that target vigilance nucleic acids. Such animals have correspondingly altered expression of vigilance polypeptides, and can thus be used to elucidate or confirm the function of vigilance molecules, or in whole-animal assays to determine of validate the physiological effect of compounds that potentially alter vigilance. The transgene may additionally comprise an inducible promoter and/or a tissue specific regulatory element, so that expression can be induced or restricted to specific cell types. Exemplary transgenic non-human animals expressing vigilance nucleic acids and nucleic acids that alter vigilance gene expression include mouse and Drosophila. Methods of producing transgenic animals are well known in the art.

The invention also provides isolated vigilance polypeptides encoded by the vigilance nucleic acid molecules of the invention. Isolated vigilance polypeptides of the invention can be used in a variety of applications. For example, isolated vigilance polypeptides can be used to generate specific antibodies, or in screening or validation methods where it is desired to identify or characterize compounds that alter the activity of vigilance polypeptides.

The isolated vigilance polypeptides of the invention can be prepared by methods known in the art, including biochemical, recombinant and synthetic methods. For example, vigilance polypeptides can be purified by routine biochemical methods from neural cells or other cells that express abundant amounts of the polypeptide. A vigilance polypeptide having any desired boundaries can also be produced by recombinant methods. Recombinant methods involve expressing a vigilance nucleic acid molecule encoding the desired polypeptide in a host cell or cell extract, and isolating the recombinant polypeptide, such as by routine biochemical purification methods described above. To facilitate identification and purification of the recombinant polypeptide, it is often desirable to insert or add, in-frame with the coding sequence, nucleic acid sequences that encode epitope tags or other binding sequences, or sequences that direct secretion of the polypeptide. Methods for producing and expressing recombinant polypeptides in vitro and in prokaryotic and eukaryotic host cells are well known in the art. Furthermore, vigilance polypeptides can be produced by chemical synthesis. If desired, such as to optimize their functional activity, stability or bioavailability, such molecules can be modified to include D-stereoisomers, non-naturally occurring amino acids, and amino acid analogs and mimetics.

Also provided are antibodies that specifically bind vigilance polypeptides encoded by the vigilance nucleic acid molecules of the invention. Such antibodies can be used, for example, in diagnostic assays such as ELISA assays to detect or quantitate the expression of vigilance polypeptides; to purify vigilance polypeptides; or as therapeutic agents to selectively target a vigilance polypeptide. Such antibodies, if desired, can be bound to a solid support, such as a chip, filter, glass slide or culture plate.

As used herein, the term “antibody” is used in its broadest sense to include polyclonal and monoclonal antibodies, as well as antigen binding fragments of such antibodies. An antibody of the invention is characterized by having specific binding activity for a vigilance polypeptide or fragment thereof of at least about 1×10⁵ M⁻¹. Thus, Fab, F(ab′)₂, Fd and Fv fragments of a vigilance polypeptide-specific antibody, which retain specific binding activity for the polypeptide, are included within the definition of an antibody. Methods of preparing polyclonal or monoclonal antibodies against polypeptides are well known in the art (see, for example, Harlow and Lane, Antibodies: A Laboratory Manual, Cold Spring Harbor Laboratory Press (1988)).

In addition, the term “antibody” as used herein includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be produced or obtained by methods known in the art, including constructing the antibodies using solid phase peptide synthesis, recombinant production, or screening combinatorial libraries consisting of variable heavy chains and variable light chains.

The invention provides diagnostic methods based on the newly identified and characterized vigilance genes described herein. In one embodiment, the invention provides a method of diagnosing a vigilance disorder in an individual. The method consists of determining a vigilance gene profile of the individual, and comparing that profile to a control profile indicative of the vigilance disorder. Correspondence between the profile of the individual and the control profile indicates that the individual has the vigilance disorder. At least one of the vigilance genes profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene containing SEQ ID NOS:2-6, 8-14, or 16-27 or modification thereof.

The methods of diagnosing vigilance disorders have numerous applications. For example, a variety of different types of sleep disorders are known, many of which are extremely common in a given population, some of which are more rare. Often individuals suffering from vigilance disorders are unaware of their disorder, or their illness has been misdiagnosed, so they are not receiving appropriate treatment. Appropriate diagnosis of the disorder will allow more effective treatments using currently available vigilance-altering compounds or methods, using compounds identified from the screens described herein, using the therapeutic methods described herein, or any combination of these treatments. Likewise, the methods of diagnosing vigilance disorders are applicable to monitoring the course of therapy for the disorder, such that appropriate modifications can be made if needed.

Furthermore, the methods of diagnosing vigilance disorders are applicable to screening for vigilance disorders among the general population, or among populations in whom sleepiness presents significant danger to the individual or to the general population (e.g. transportation workers, individual operating heavy machinery, and the like). Likewise, the methods of diagnosing vigilance disorders can be used in conjunction with diagnosis or prognosis of an associated medical or psychiatric condition. Additional useful applications of the diagnostic methods of the invention can be determined by those skilled in the art.

As used herein, the term “vigilance disorder” refers to any condition that disturbs the normal sleep and wake patterns of an individual. A vigilance disorder can have a genetic or familial basis; can have a psychiatric or medical basis; can be induced by substances including medications and drugs; or can have any combination of these underlying causes. Exemplary vigilance disorders include, but are not limited to, various forms of insomnia, hypersomnia, narcolepsy, parasomnias, sleepwalking disorder, sleep apnea, restless legs syndrome (RLS) and fatal familial insomnia. A variety of vigilance disorders in humans are described in Diagnostic and Statistical Manual of Mental Disorders, 4th Edition (1994), published by the American Psychiatric Association.

Appropriate laboratory animal models of human vigilance disorders of interest are known in the art or can readily be developed by transgenic and knockout methods that alter expression or activity of vigilance genes, or by pharmacological, surgical or environmental manipulation. For example, as described in Chemelli et al., Cell 98:409-412 (1998), orexin (hypocretin) knockout mice, as well as canarc-1 mutant dogs, are animal models of human narcolepsy. Additionally, Michaud et al., Arch. Int. Pharmacodyn. Ther. 259:93-105 (1982), describes a rat model of insomnia that is applicable for pharmacological research. Panckeri et al., Sleep 19:626-631(1996), describes that the English bulldog is a natural model of sleep-disordered breathing (SDB), and canine models of obstructive sleep apnea are described in Kimoff et al., J. Appl. Physiol. 76:1810-1817 (1994).

The diagnostic methods of the invention can also advantageously be used to characterize previously unrecognized vigilance disorders, or newly categorize vigilance disorders, based on characteristic patterns of expression or activity of vigilance genes. Such newly characterized or categorized disorders are also encompassed by the term “vigilance disorder.” The diagnostic methods of the invention can also be advantageously used to identify the specific vigilance genes most closely associated with, and thus likely to play a causative role, in particular vigilance disorders. Such genes are targets for modulation by gene therapy methods or by selective targeting of the encoded product with therapeutic compounds.

In a further embodiment of the diagnostic methods of the invention, there is also provided a method of determining vigilance level in an individual. The method consists of determining a vigilance gene profile of the individual, and comparing that profile to a control profile indicative of a predetermined vigilance level. Correspondence between the profile of the individual and the control profile indicates that the individual exhibits the predetermined vigilance level. At least one of the vigilance genes profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene containing SEQ ID NOS:2-6, 8-14, or 16-27 or modification thereof.

Physiological correlates of depth of sleep (e.g. stages of REM and non-REM sleep) and degree of alertness in laboratory animals and humans are well known in the art. As described above, corresponding behavioral correlates of sleep and wake states are now also known in invertebrates. Thus, control vigilance gene profiles can be established from invertebrates, other animals or humans that are indicative of the range of potential vigilance levels, from highly alert, to drowsy, to lightly asleep, to deeply asleep, to unconscious. Control vigilance gene profiles can also be established indicative of the transition between normal sleep and wake or between normal wake and sleep; indicative of sleep deprivation or indicative of sleep rebound. Control vigilance gene profiles can also be established indicative of the quality or quantity of sleep or wake in the previous sleep or wake period. Thus, in a test individual, a vigilance gene profile can be determined, and compared to any of the established control profiles to determine the vigilance level of that individual.

The methods of the invention for determining vigilance level in an individual are advantageous over previous methods of determining vigilance level (e.g. cognitive tests, arousal assays, EEG) in that vigilance gene profiles are precise molecular fingerprints characteristic of every possible vigilance level and state of interest. Accordingly, the precise effect of anaesthesia, medications (including vigilance-altering medications), medical procedures, stress, environmental conditions, and the like, on vigilance level in an individual can be readily determined by a simple assay that can be performed on either sleeping or awake individuals. Such information is valuable, for example, in choosing an appropriate course of medical treatment for a patient that will avoid undesirable effects on vigilance, such as disrupting restorative sleep, decreasing daytime alertness, or causing excessive sleep rebound. Furthermore, should it be preferable to continue treatment with a medication that causes such undesirable side effects, by knowing which vigilance genes are undesirably altered, a clinician can determine which vigilance-altering therapeutic should concurrently, previously or subsequently be administered to counteract the medication to restore more normal activity or expression of those vigilance genes, and thus reduce or eliminate the undesirable side effects.

As used herein, the term “vigilance gene profile” refers to any read-out that provides a qualitative or quantitative indication of the expression or activity of a single vigilance gene, or of multiple vigilance genes. A vigilance gene profile can, for example, indicate the expression or activity of one, or of least 2, 5, 10, 20, 50, 100 or more vigilance genes. A vigilance gene profile can, for example, indicate the expression or activity in mammals of mammalian homologs of one or more vigilance genes identified as such from the invertebrate screening assays described herein, such as Fas, BiP, Cyp4e2, AANAT1 (Dat), Ddc, or a gene containing any of SEQ ID NOS:2-6. A vigilance gene profile can alternatively or additionally indicate the expression or activity of one or more vigilance genes identified as such from mammalian studies described herein, such as the homolog in that mammal of Cytochrome P450, AA117313, aryl sulfotransferase IV, human breast tumor autoantigen, KIAA313, E25, or a gene containing any of SEQ ID NOS:8-14 and 16-27. A vigilance gene profile can additionally indicate the expression or activity of one or more vigilance genes identified as such from published mammalian studies described above, including NGFI-A, NGFI-B, rlf, Arc, JunB, IER5, Cytochrome oxidase C subunit 1, Cytochrome oxidase C subunit IV, NADH dehydrogenase subunit 2, 12S rRNA F1-ATPase subunit alpha, Ng/RC3, bone morphogenetic protein 2, GRP78, BDNF, IL-1β, dendrin, Ca⁺⁺/calmodulin-dependent protein kinase II α-subunit, orexin, orexin receptor, and PRNP.

It is estimated that at least about 1% of genes in animals are vigilance-modulated. Thus, a vigilance gene profile can indicate expression or activity of one, a few, many, or all of these vigilance genes. A vigilance gene profile can also indicate expression or activity of other genes that not previously characterized as vigilance genes, which may then be determined to be vigilance genes.

A “vigilance gene profile” can be, for example, a quantitative or qualitative measure of expression of mRNA expressed by a vigilance gene. A variety of methods of detecting or quantitating mRNA expression have been described above in connection with invertebrate screening assays and include, but are not limited to, Northern or dot blot analysis, primer extension, RNase protection assays, differential display, reverse-transcription PCR, competitive PCR, real-time quantitative PCR (TaqMan PCR), and nucleic acid array analysis.

A “vigilance gene profile” can also be a quantitative or qualitative measure of expression of polypeptides encoded by vigilance genes. Methods of detecting or quantitating protein expression have been described above in connection with invertebrate screening assays, and include, but are not limited to, immunohistochemistry, immunofluorescence, immunoprecipitation, immunoblot analysis, and various types of ELISA analysis, including ELISA analysis using arrays of vigilance-polypeptide specific antibodies bound to solid supports. Additional methods include two-dimensional gel electrophoresis, MALDI-TOF mass spectrometry, and ProteinChip™/SELDI mass spectrometry technology.

A “vigilance gene profile” can also be a direct or indirect measure of the biological activity of polypeptides encoded by vigilance genes. A direct measure of the biological activity of a vigilance polypeptide can be, for example, a measure of its enzymatic activity, using an assay indicative of such enzymatic activity. An indirect measure of the biological activity of a polypeptide can be its state of modification (e.g. phosphorylation or glycosylation) or localization (e.g. nuclear or cytoplasmic), where the particular modification or localization is indicative of biological activity. A further indirect measure of the biological activity of a polypeptide can be the abundance of a substrate or metabolite of the polypeptide, such as a neurotransmitter, where the abundance of the substrate or metabolite is indicative of the biological activity of the polypeptide. Appropriate assays for measuring enzyme activity, polypeptide modifications, and substrates and metabolites or vigilance polypeptides, will depend on the biological activity of the particular vigilance polypeptide.

The appropriate method to use in determining a vigilance gene profile can be determined by those skilled in the art, and will depend, for example, on the number of vigilance genes being profiled; whether the method is performed in vivo or in a sample; the type of sample obtained; whether the assay is performed manually or is automated; the biological activity of the encoded vigilance polypeptide; the abundance of the transcript, protein, substrate or metabolite being detected; and the desired sensitivity, reproducibility and speed of the method.

A vigilance gene profile can be established in vivo, such as by diagnostic imaging procedures using detectably labeled antibodies or other binding molecules, or from a sample obtained from an individual. As changes in vigilance gene expression in the brain are likely to be most relevant to regulation of the sleep-wake cycle, appropriate samples can contain neural tissue, cells derived from neural tissues, or extracellular medium surrounding neural tissues, in which vigilance polypeptides or their metabolites are present. Thus, an appropriate sample for establishing a vigilance gene profile in humans can be, for example, cerebrospinal fluid, whereas in laboratory animals an appropriate sample can be, for example, a biopsy of the brain.

However, expression of vigilance genes can also be modulated during the sleep-wake cycle in other tissues than neural tissue, and vigilance polypeptides or their metabolites can be secreted into bodily fluids. In particular, in the case of genetic vigilance disorders, including monogenic vigilance disorders, any alteration in vigilance gene expression or function will be manifested in every cell in the body that normally expresses the vigilance gene. Thus, a vigilance gene profile can be established from any convenient cell or fluid sample from the body, including blood, lymph, urine, breast milk, skin, hair follicles, cervix or cheek. Additionally, cells can readily be obtained using slightly more invasive procedures, such as punch biopsies of the breast or muscle, from the bone marrow or, during surgery, from essentially any organ or tissue of the body.

The diagnostic methods of the invention are practiced by determining a vigilance gene profile of an individual, and comparing the profile of that individual to a control profile. As used herein, the term “individual” refers to any mammalian individual, such as a human, a veterinary animal, or a laboratory animal. The control profiles, which as described above include profiles established from invertebrates or of “individuals” can have be determined previously, simultaneously or subsequently to determining the vigilance gene profile of the test individual.

In the diagnostic methods described herein, correspondence between the vigilance gene profile of the individual and the control profile is evaluated. As used herein, the term “correspondence” refers to a significant degree of similarity, including identity, in pattern or amount of expression or activity between the vigilance gene profile in the individual and the control profile. The degree of similarity or identity required to establish correspondence can be determined by those skilled in the art, and will depend on several factors including the number of vigilance genes being examined; the usual range of variation in expression or activity of the vigilance genes between conditions or individuals; the relevance of a particular vigilance gene to the vigilance disorder or vigilance level being evaluated; and the sensitivity of the assay being used. In general, the term “correspondence” refers to a vigilance gene profile that is more similar to the control vigilance profile than to a vigilance profile that is indicative of a different vigilance disorder, level or state than the control vigilance profile.

Those skilled in the art understand that the methods described above for diagnosing vigilance disorders and determining vigilance level can readily be applied to methods of screening for novel vigilance-altering compounds; to methods of validating the efficacy of vigilance-altering compounds identified by other methods, such as by the invertebrate screening methods described above; to methods of determining effective dose, time and route of administration of known vigilance-altering compounds; to methods of determining the effects of vigilance-altering compounds on homeostatic regulation of vigilance; to methods of determining the molecular mechanisms of action of known vigilance-altering compounds; and the like. Such methods can be performed in laboratory animals, such as mice, rats, rabbits, dogs, cats, pigs or primates, in veterinary animals, or in humans.

Thus, in one embodiment, the invention provides a method of determining the efficacy of a compound in ameliorating a vigilance disorder. The method consists of administering the compound to an individual having a vigilance disorder, and determining an effect of the compound on the vigilance gene profile of the individual. A compound that modulates the vigilance gene profile of the individual to correspond to a normal vigilance profile indicates that the compound is effective in ameliorating the vigilance disorder. At least one of the vigilance genes profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene containing SEQ ID NOS:2-6, 8-14, or 16-27 or modification thereof.

As used herein, the term “ameliorating” is intended to include preventing, treating, curing, and reducing the severity of the vigilance disorder. Those skilled in the art understand that any degree of reduction in severity of a vigilance disorder can improve the health or quality of life of the individual. The effect of the therapy can be determined by those skilled in the art, by comparison to baseline values for vigilance properties affected in the disorder.

In another embodiment, the invention provides a method of determining the efficacy of a compound in modulating vigilance. The method consists of administering the compound to an individual, and determining an effect of the compound on the vigilance gene profile of the individual. A compound that modulates the vigilance gene profile indicates that the compound modulates vigilance. At least one of the vigilance genes profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene containing SEQ ID NOS:2-6, 8-14, or 16-27 or modification thereof.

The vigilance genes to profile can be determined by those skilled in the art, depending on the type of vigilance-altering compound it is desired to identify or characterize. For example, it may be advantageous to examine the effect of a compound primarily on single genes whose causitive role in vigilance has been established, including Dat, Ddc, orexin, orexin receptor and PRNP; or only or primarily on those vigilance genes whose expression or activity is upregulated during sleep; or only or primarily on those vigilance genes whose expression or activity is upregulated during wake; or only or primarily on those genes whose expression is modulated during sleep rebound, during sleep-wake transition, or in the period following restorative or disrupted sleep.

The compounds so identified that alter vigilance gene profile can, for example, enhance vigilance, decrease vigilance and/or alter or maintain a homeostatically regulated property of vigilance such as period of sleep rebound, latency to sleep, rate of sleep-wake transition, or vigilance properties in the period following changes in sleep or wake, as described above in relation to invertebrate screening methods. The effect of these compounds on any of these vigilance properties can be corroborated, or further evaluated, in either invertebrates or mammals. The effect of the compounds on learning or memory in invertebrates or mammals can also be assessed. Compounds that beneficially alter one or a combination of vigilance properties can be administered as therapeutics to humans and veterinary animals.

Once genes associated with vigilance disorders and vigilance levels are identified, the expression or activity of such genes in humans or veterinary animals can be selectively targeted in order to prevent or treat the vigilance disorder, or to beneficially alter vigilance level, state or a homeostatically regulated property of vigilance. The diagnostic, screening and validation methods of the invention are useful in determining appropriate genes to target and appropriate therapeutic compounds to use for a particular indication. Additional vigilance genes can be identified by the methods described herein or by other methods, including differential display, arrays, and other forms of expression or activity analysis in invertebrates and mammals; genetic methods, such as by randomly or specifically targeting genes in model organisms such as Drosophila or mouse, or by mapping genes associated with vigilance disorders or altered vigilance properties; or from screens for genes associated with other behaviors or molecular pathways that are subsequently determined to be associated with vigilance.

Thus, in one embodiment, the invention provides a method of ameliorating a vigilance disorder in an individual. The method consists of administering to an individual having a vigilance disorder an agent that modulates the vigilance gene profile of the individual to correspond to a normal vigilance gene profile. At least one of the vigilance genes profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene containing SEQ ID NOS:2-6, 8-14, or 16-27 or modification thereof. In one embodiment, the vigilance gene modified is one of the recited genes.

In a further embodiment, the invention provides a method of modulating vigilance level in an individual. The method consists of administering to an individual an agent that modulates the vigilance gene profile of the individual. At least one of the vigilance genes profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene containing SEQ ID NOS:2-6, 8-14, or 16-27 or modification thereof. In one embodiment, the vigilance gene modified is one of the recited genes.

The therapeutic methods of the invention involve determining the effect of the agent on vigilance gene profile. Thus, the therapeutic methods of the invention are not intended to encompass administration of vigilance-altering drugs which inherently may modulate vigilance gene expression or activity, in the absence of a determination that such drugs predictably modulate vigilance gene profile. The effect of the therapeutic agent on vigilance gene profile in the particular individual in whom the agent is administered need not be determined, however, if the effect of the therapeutic agent on vigilance gene profile in other individuals has previously been established, and such effect on vigilance gene profile can be shown to be reproducible across individuals, of course, it is understood that the vigilance gene profile of the individual can, if desired, be determined prior to administration of the therapeutic agent, and/or monitored during the course of therapy, using modifications of the diagnostic methods described herein.

A variety of therapeutic agents can be used to modulate vigilance gene profile in individuals having a vigilance disorder or in whom alteration of vigilance level is desired. Agents can be determined or designed to alter vigilance gene expression or activity by a variety of mechanisms, such as by directly or indirectly increasing or decreasing the expression of a vigilance gene. For example, a therapeutic agent can directly interact with the vigilance gene promoter; can interact with transcription factors that regulate vigilance gene expression; can bind to or cleave the vigilance gene transcript (e.g. antisense oligonucleotides or ribozymes); can alter half-life of the transcript; or can be an expressible vigilance gene itself. A therapeutic agent can also act by increasing or decreasing activity of one or more encoded vigilance polypeptides. For example, the agent can specifically bind to a vigilance polypeptide and alter its activity or half-life; can bind to a substrate or modulator of a vigilance polypeptide; or can be the vigilance polypeptide or active portion thereof.

The type of agent to be used can be determined by those skilled in the art, and will depend, for example, on factors such as the severity of the disorder; the time period over which correction of the disorder or alteration of the vigilance level is desired; the cellular location of the vigilance molecule to be targeted; whether the agent is administered in a clinical setting or by the individual; and when during the sleep-wake cycle the agent is administered. In general, therapeutic agents useful in the methods of the invention include “compounds,” as described above, including small molecules, and gene therapy molecules.

Therapeutic agents can be formulated in pharmaceutical compositions in such a manner to ensure proper distribution in vivo. For example, the blood-brain barrier (BBB) excludes many highly hydrophilic compounds. To ensure that the therapeutic agents of the invention cross the BBB, they can be formulated, for example, in liposomes, or chemically derivatized. Methods of introduction of a therapeutic agent of the invention include, but are not limited to, intradermal, intramuscular, intraperitoneal, intravenous, subcutaneous, oral, intranasal, intraspinal and intracerebral routes. An agent can also appropriately be introduced by rechargable or biodegradable polymeric devices, which provide for the slow release or controlled delivery of drugs. Appropriate formulations, routes of administration and dose of a therapeutic agent can be determined by those skilled in the art.

If desired, the therapeutic agents of the invention can include gene therapy molecules that modulate vigilance gene expression or activity, including genes encoding vigilance polypeptides or active or inhibitory portions thereof; genes expressing antisense molecules that block expression of vigilance genes; and genes expressing ribozymes that target vigilance genes. Such methods are advantageous in ameliorating monogenic vigilance disorders or for providing long-lasting effects to an individual. Methods of introducing and expressing genes in animals, including humans, are well known in the art.

Gene therapy methods can be performed ex vivo, wherein cells (e.g. hematopoietic cells, including stem cells) are removed from the body, engineered to express a vigilance polypeptide, and returned to the body. Gene therapy methods can also be performed in situ, in which an expressible nucleic acid molecule is placed directly into an appropriate tissue, such as the brain or CNS, by a direct route such as injection or implantation during surgery. Gene therapy methods can also be performed in vivo, wherein the expressible nucleic acid molecule is administered systemically, such as intravenously. Appropriate vectors for gene therapy can be determined by those skilled in the art for a particular application of the method, and include, but are not limited to, retroviral vectors (e.g. replication-defective MuLV, HTLV, and HIV vectors); adenoviral vectors; adeno-associated viral vectors; herpes simplex viral vectors; and non-viral vectors. Appropriate formulations for delivery of nucleic acids can also be determined by those skilled in the art, and include, for example, liposomes; polycationic agents; naked DNA; and DNA associated with or conjugated to targeting molecules (e.g. antibodies, ligands, lectins, fusogenic peptides, or HIV tat peptide). Gene therapy methods, including considerations for choice of appropriate vectors, promoters, formulations and routes of delivery, are reviewed, for example, in Anderson, Nature 392:25-30 (1998).

It is understood that modifications which do not substantially affect the activity of the various embodiments of this invention are also included within the definition of the invention provided herein. Accordingly, the following examples are intended to illustrate but not limit the present invention.

### EXAMPLE I

### Behavioral Correlates of Sleep in Drosophila

This example shows that Drosophila exhibits sleep that is similar to mammalian sleep, as evidenced by the main behavioral criteria for sleep, namely sustained behavioral quiescence (rest), increased arousal threshold, and increased sleep following prolonged waking (homeostatic regulation).

In order to monitor fly behavior, five-day old virgin female Canton-S Drosophila melanogaster were cultured at 25° C., 50-60% humidity, 12 hr:12 hr light:dark cycle, on brewer's yeast, dark corn syrup and agar food, following procedures modified from J. Bennett and D. L. van Dyke, Dros. Inform. Serv. 46:160 (1971). Continuous, high-resolution measurement of fly behavior was achieved using an ultrasound activity monitoring system shown in FIG. 1A. Briefly, a 44 kHz standing wave was passed across an independent enclosure containing a single fly. An integrated circuit sampled a portion of each wave as a function of the transmit signal and compared it to the output from the receive signal for the same time-window. When the fly moved its mass within the field, it perturbed the standing wave and the resulting difference was counted as a movement. The output was sampled by a PC at 200 Hz, the data were summed in 2-sec bins and stored for later processing. This system detects very small movements in Drosophila's behavioral repertoire, including fine movements of the head, wings, and limbs.

In order to validate the output of the ultrasound activity monitoring system, five behaviors were visually scored in 2-sec bins by an observer blind to the output of the ultrasound system on 18 independent trials for a total of 8 h. The correspondence rates for specific states were as follows: Locomoting=99%, Inactive=97%, Grooming anterior limbs=94%, Grooming posterior limbs=98%, and Eating=97%. This correspondence rate is similar to that found between measures of activity and polysomnography in humans. A representative validation trial lasting 60 min is shown in FIG. 1B, and indicates that the ultrasound output and visual observation are in good agreement.

As shown in FIG. 1C, using the ultrasound activity monitoring system, female flies maintained on a 12:12 light dark cycle were active throughout the light period (horizontal white bar) and exhibited few periods of sustained inactivity. In contrast, during the dark period (horizontal black bar) there were extended bouts of quiescence. Based on pilot studies, rest was defined as uninterrupted behavioral quiescence lasting for at least 5 min. Greater than 90% of rest occurred during the dark period, as shown in FIG. 1C.

To monitor rest-activity patterns in large numbers of flies, an infrared Drosophila Activity Monitoring System was used (Trikinetics; described in M. Hamblen et al., J. Neurogen. 3:249 (1986)). To validate the system, flies were visually monitored for a total of 17.75 h (n=7). The number of times the fly crossed the infrared beam was counted in 5-minute bins. Flies were awake but did not cross the beam in 5 out of 213 bins (miss rate=2.35%). The results obtained with the infrared activity monitoring system demonstrated robust circadian organization of activity and showed good correspondence with the ultrasound monitoring system.

In order to determine whether periods of rest are associated with increased arousal thresholds, flies were subjected to vibratory stimuli of increasing intensity (0.05 g, 0.1 g, and 6 g). In these experiments, flies were placed in glass tubes (65 mm in length, 5 mm I.D.) maintained on a hard plastic platform above a Grass speaker. The output of the speaker was controlled via a Beckman signal generator and the resulting vibration of the platform was measured with an accelerometer. Each fly received a stimulus each hour (total of 8 stimuli) of constant intensity. The behavioral state at the time of stimulus delivery and the ensuing response were videotaped and scored off-line.

Flies that had been behaviorally awake readily responded to intensities of 0.05 g and 0.1 g (90% of trials). Flies that had been behaviorally quiescent for 5 minutes or more rarely showed a behavioral response to these stimuli (˜20% of trials; p<0.001, χ²). However, when the intensity of the stimulus was increased to 6 g, all flies quickly responded regardless of behavioral state (p>0.1, χ²).

These results indicate that, like sleep in mammals, sustained periods of quiescence in Drosophila are characterized by increased arousal thresholds.

It was next investigated whether the amount of rest in Drosophila is homeostatically regulated. Under baseline conditions the amount of rest during the light period was quite low (FIG. 2A, open circles). Flies (n=24) were deprived of rest individually by gentle tapping of their containers at rest onset (about 4 stimuli/min) for 12 h during the dark period. Efforts were made to avoid disturbing the flies if they were eating or grooming. During the first 12 h of the following light period, rest-deprived flies (FIG. 2A, black squares; p<0.001, Wilcoxon signed-ranks test for matched pairs) exhibited a seven-fold increase in rest compared to baseline.

Additionally, an automated system was used to rest-deprive large numbers of flies. Only flies that were active (indicated by the number of infrared crossings) for at least 66% of the light period and inactive (no infrared crossings) for at least 66% of the dark-period were studied. Rest deprivation was achieved by placing glass tubes, containing individual flies, into a cylinder that was rotated in a hybridization oven (Hybaid) at 10 revolutions/minute. At the nadir of the arc the tubes would be carried to the apex and dropped 2.5 cm. Note that flies were not forced to walk throughout each cycle.

Automated rest deprivation for 12 h during the dark period resulted in a three-fold increase in rest over baseline values during the first 6 h of the following light period (mean of 10 independent experiments, n=286, FIG. 2A, gray triangles; all z>3.1, p<0.001). In the first 24 h following manual rest deprivation, flies recovered 50% of the rest that was lost, a value comparable to the sleep rebound seen in mammals following short-term sleep deprivation.

To investigate whether the homeostatic regulation is separable from circadian factors, per⁰¹ mutant flies, which are arrhythmic under constant darkness, were examined. Under constant darkness, per⁰¹ flies had the same amount of rest as under light-dark conditions (p>0.5), but the amount of rest was evenly distributed across the 24 hours (open circles). Twelve hours of automated rest deprivation in constant darkness resulted in a significant increase in rest during the first 6 h of recovery (black squares) compared to baseline (n=25, p<0.001). Since rest is evenly distributed in per⁰¹ flies, rest deprivation eliminated only about 50% of daily rest, compared to 90% in wild-type flies.

Recordings with the ultrasound system showed that the rest rebound after deprivation was characterized by actual immobility and not simply an increase of stationary waking activities, such as eating or grooming, that may result in reduced infrared beam crossing. Moreover, the amount of activity during the deprivation was not correlated with the size of the rest rebound, indicating that the increase in rest was not due to levels of prior activity (FIG. 2B, inset). Consistent with this, when flies were stimulated in the apparatus for 12 h during the light period, rest not only failed to increase, but was actually reduced by 16+/−4% during the first 6 h of recovery (FIG. 2B, compare gray diamonds (rest deprived) with open circles (baseline)). Thus, the increase in rest is not due to physical exhaustion induced by forced activity.

Additional controls were used to validate the infrared system. Flies deprived of food for 12 h during the dark period and given food during the following light period showed no change in the number of infrared crossings. This result indicated that eating was not miscoded as rest. Food deprivation has been shown to increase activity in Drosophila (Connolly, Nature 209:224 (1966)) and waking in mammals (Jacobs et al., Exp. Neural. 30:212 (1971)). It was determined that food deprivation for 1 day increased waking by 50% in Drosophila. In addition, dusting flies with Reactive Yellow, as described in Phillis et al., Genetics 133:581 (1993), increased grooming behavior by 72% but did not reduce the number of infrared crossings. This result indicated that grooming was not miscoded as rest.

In additional experiments it was determined that male flies obtain 70% of their daily rest during the dark period and exhibit an additional rest peak between 03.00 and 07.00 during the light period. Rest deprivation using the automated system revealed that both nighttime rest and rest during the day are homeostatically regulated.

These results indicate that rest in Drosophila, like sleep in mammals, is under homeostatic control.

### EXAMPLE II

### Age-Dependence of Sleep in Drosophila

This example shows that Drosophila sleep, like mammalian sleep, exhibits age dependence. This example also shows that homeostatic regulation of sleep is preserved in older flies.

In mammals, sleep is prominent in the very young, stabilizes during adolescence and adulthood, and declines during old age (see Stone, Clin. Ger. Med. 5:363 (1989); Bliwise, in Principles and Practice of Sleep Medicine, Kryger et al. Eds. (Saunders, Philadelphia, 2^{nd} ed., 1994), chap. 3; Dijk et al., J. Physiol. 516:611 (1999)). To determine whether sleep in Drosophila follows a similar pattern, Drosophila rest was assayed at various days after eclosion using the infrared system.

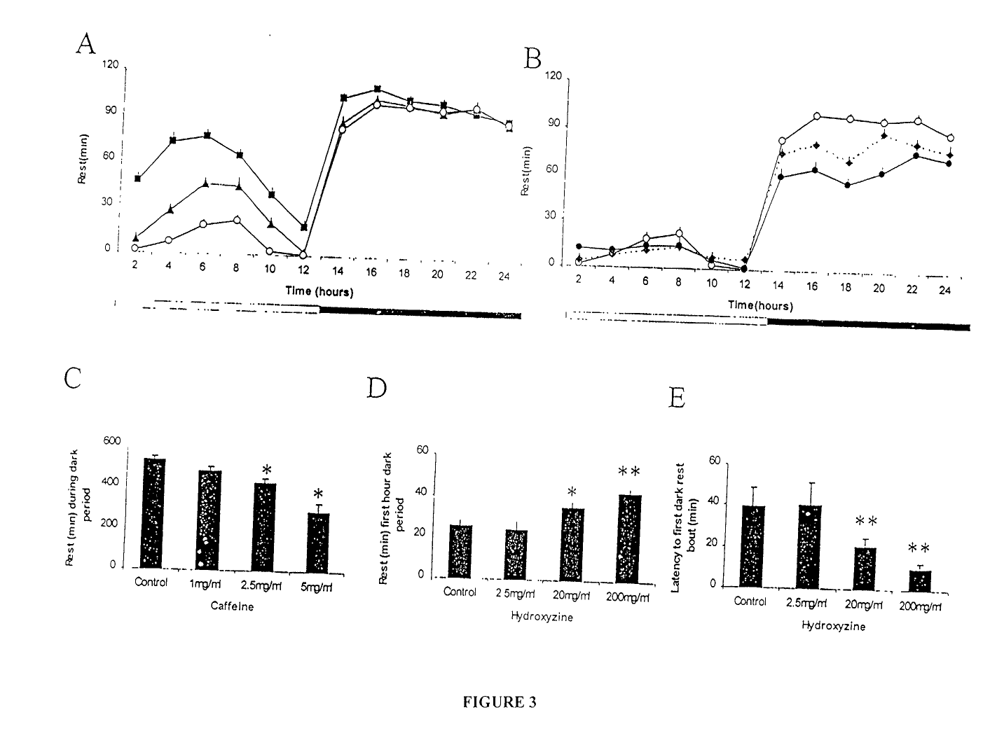

As shown in FIG. 3A, on the first full day after eclosion (black squares) rest was pronounced, decreased on day 2 (gray triangles), and reached stable adult values by day 3 (open circles; p<0.001; ANOVA, Bonferroni correction). As shown in FIG. 3B, as the flies aged the amount of rest during the night began to decline (gray diamonds, 16 days of age) and was significantly below that found in young adults (open circles, 3 days of age) by 33 days of age (black circles, p<0.001).

These results indicate that rest in Drosophila follows a similar age-dependent pattern as sleep in mammals.

Several studies indicate that the homeostatic regulation of sleep is preserved in older humans (see Stone, Clin. Ger. Med. 5:363 (1989); Bliwise, in Principles and Practice of Sleep Medicine, Kryger et al. Eds. (Saunders, Philadelphia, 2^{nd} ed., 1994), chap. 3; Dijk et al., J. Physiol. 516:611 (1999)). When 33 day old flies were deprived of rest they exhibited a rest rebound which was similar to that seen in young flies.

These results indicate that homeostatic regulation of rest is preserved in older flies, as it is in older mammals.

### EXAMPLE III

### Pharmacological Modulation of Sleep in Drosophila

This example shows that pharmacological compounds that modulate mammalian vigilance level also modulate fly vigilance level.

Sleep in mammals is modulated by several classes of drugs that act as stimulants or hypnotics. For example, caffeine increases wakefulness and motor activity, while antihistamines reduce sleep latency (Yanik et al., Brain Res., 403:177 (1987)). While the mutagenic effects of caffeine in the fly are well-studied (e.g. Legator et al., J. Environ. Sci. Hlth. 13: 135 (1979); Dudai, Israel J. Med. Sci. 15:802 (1979); Itoyamaet al., Cytobios. 83:245 (1995); Nassel, Microsc. Res. Tech. 44:121 (1999) ), little is known about its behavioral effects.

Drugs (caffeine or hydroxyzine) dissolved in food were continuously available to flies beginning in the final hour of the light period. As shown in FIG. 3C, when flies were given caffeine, the amount of rest during the dark period decreased in a dose-dependent fashion (n=36/dose, *, p<0.0001) and motor activity increased.

Histamine has been shown to be a neurotransmitter in the central and peripheral nervous system of the fly (Nassel, Microsc. Res. Tech. 44:121 (1999)). When flies were given hydroxyzine, an antagonist of the Hl histamine receptor, rest during the first hour of the dark period was increased in a dose-dependent manner (FIG. 3D), and latency to first dark period rest was decreased (FIG. 3E) (n=40/dose, *, p=0.056; **, p<0.001). The increase in rest was not associated with a general impairment of fly behavior. The activity per waking minute was unchanged during the dark period (both during the first hour and the subsequent hours). The total amount of activity during the light period was also unchanged. Furthermore, responsiveness to arousing stimuli was preserved.

Thus, two agents that modulate waking and sleep in mammals also modulate vigilance states in Drosophila.

### EXAMPLE IV

### Molecular Correlates of Sleep in Drosophila

This example shows that Drosophila gene expression is modulated by vigilance state, in a similar manner as it is in mammals.

Recently, several genes have been identified whose expression in the rat brain changes in relation to sleep and waking (see Cirelli et al., Mol. Brain Res. 56:293 (1998); Cirelli et al., Ann. Med. 31:117 (1999); Cirelli et al., Sleep 22(S):113 (1999)). In order to determine whether there are any molecular changes associated with the rest-activity cycle in the fly, gene expression in Drosophila was systematically screened using mRNA differential display as well as a targeted approach with RNase protection assays (RPA) to search for specific genes.

mRNA differential display and RPA were performed as in Cirelli et al., Mol. Brain Res. 56:293 (1998), with the following modifications. For differential display, reverse transcription was performed with 0.55 pg of pooled total RNA from fly heads (n=20). Two independent pools were reverse-transcribed per condition. PCR reactions were performed in duplicate for each pool. One hundred and four combinations of primers were used. For RPA, 1-2 μg of total RNA from pooled fly heads (n=60) were used. The amount of sample RNA was normalized using a riboprobe specific for ribosomal protein rp49.

RNA was extracted from whole heads of flies that (I) had been spontaneously resting for 3 h during the dark period; (ii) had been rest deprived for 3 h and were collected at the same circadian time, or (iii) had been spontaneously awake for 3 h during the light period (see FIG. 4A). This allowed distinguishing between changes in gene expression associated with behavioral state and those associated with circadian time or with stimulation.

The behavioral state was determined individually for each fly; only flies that satisfied specific criteria were selected for analysis. In particular, a fly was considered to be awake if it was active for at least 90% of the 3-hour light period and 100% of the hour before sacrifice. A fly was resting if it was inactive for at least 66% of the 3-hour dark period and 100% of the hour before sacrifice. Only about 60-70% of the flies examined satisfied these criteria. It should be noted that failure to specifically identify rest and waking, as has been done in circadian screens, results in samples containing a mixture of behavioral states.

Similar to what has been shown in rat, it was determined that about 1% of the transcripts examined in Drosophila were modulated by behavioral state. Out of an estimated 5,000 RNA species screened, 54 were expressed at higher levels during waking than during rest and 28 were higher during rest.

Several transcripts (46) showed a prominent circadian, but not state-dependent, modulation (Van Gelder et al., Curr. Biol. 5: 1424 (1995)). For example, a transcript designated “Circadian” was increased by 400% in the dark conditions (both rest and rest deprivation) with respect to the light condition (waking). This transcript did not correspond to any known sequence. An additional gene which showed a circadian, but not state-dependent, modulation was Drosophila fos (Perkins et al., Genes Dev. 4:822 (1990)). D-fos was expressed at higher levels during the dark hours, irrespective of behavioral state. By contrast, in rat (and cat) c-fos is high during waking and low during sleep, irrespective of circadian time (Pompeiano et al., J. Sleep Res. 3:80 (1994)). In the rat suprachiasmatic nucleus, c-fos expression is modulated in a circadian way by light (Schwartz et al., Sem. Neurosci. 7:53 (1995)). It should be noted that the transcriptional activity of CREB, which is necessary for fos induction, is also higher during the dark hours in Drosophila (Belvin et al., Neuron 22:777 (1999)).

An example of a transcript whose expression was higher after periods of rest was designated “Rest”. As confirmed using RPA, this mRNA was 45% higher in rest than in rest deprivation. None of the rest-related transcripts matched any published sequence, similar to the results in the rat.

By contrast, several known genes were identified that were expressed at higher levels during waking than during rest, irrespective of circadian time (p<0.1, ANOVA). One, with high homology to Fatty acid synthase (Fas), was increased after 3 h of spontaneous waking or rest deprivation compared to rest (by 50% and 88%, respectively, using RPA, as shown in FIG. 4B, top). This sequence matched a Drosophila Pl Clone (AC005554). Subsequent analysis using Genescan indicated that the sequence matched a proposed peptide that had 49% homology with rat FAS.

Since Fas expression had not been studied in the fly, in situ hybridization with digoxigenin-labeled probes was performed as described in Aronstein et al., Neuroscience 2:115 (1996). In situ analyis indicated that the Fas transcript is expressed throughout the fly brain, including the optic lobes, but not in the eye. Although the role of this enzyme in the fly brain not clear, fatty acids are increasingly being recognized as modulators of neural activity (see Clark, Evolution 44:637 (1990); Yehuda et al., Peptides 19:407 (1998)).

Significantly, several genes were identified that were upregulated during waking vs. rest in the fly that corresponded to genes upregulated during waking vs. sleep in the rat, irrespective of circadian time. In the rat, mitochondrial genes, including Cytochrome oxidase C, subunit I, show a rapid increase in expression during the first few hours of waking (Cirelli et al., Mol. Brain Res. 56, 293 (1998); Cirelli et al., Ann. Med. 31:117 (1999); Cirelli et al., Sleep 22(S):113 (1999) and FIG. 4C, bottom). In Drosophila, mRNA levels of Cytochrome oxidase C, subunit I, also show a rapid increase during the first few hours of waking with respect to rest (FIG. 4C, top). Such rapid changes in the expression of the mitochondrial genome are thought to represent a local response of nervous tissue to the increased metabolic requirements of waking (Wong-Riley et al., Neuroscience 76, 1035 (1997); Cirelli et al., Mol. Brain Res. 56:293 (1998)).

Cytochrome P450 (Cyp4e2), a member of a large family of detoxifying enzymes (Dunkov et al., Mol. Gen. Genet. 251:290 (1996)), was also increased in waking and rest deprivation with respect to rest by 77% and 99%, respectively (FIG. 4B, bottom). A related cytochrome P450 (Cyp4F5) was upregulated after periods of waking in rat cerebral cortex, as demonstrated by using gene discovery arrays and RPA (Rat Atlas cDNA 1.2 expression array (Clontech)).

BiP is a chaperone protein localized in the endoplasmic reticulum that assists in the folding and assembly of newly synthesized secretory and transmembrane proteins. BiP may also serve as a calcium buffer (Pahl et al., Physiol. Rev. 79:683 (1999)). In Aplysia, the homologue of BiP is upregulated within 3 h of behavioral training and is thought to promote the structural changes necessary for the establishment of long-term memory (Kuhl et al., J. Cell Biol. 119:1069 (1992)). FIG. 4D (bottom) shows that, in the rat, BiP mRNA is expressed at higher levels after periods of spontaneous waking and sleep deprivation (8 h) than after periods of sleep. A similar pattern is found in Drosophila (FIG. 4D, top). After spontaneous waking and rest deprivation (3 h), BiP mRNA exhibits a 2-fold and 3-fold increase above resting values, respectively.

It was also determined that mRNA levels of arylalkyamine N-acetyl transferase (Dat) were increased by 48% after 2-3 h of waking compared to rest. This enzyme, which is found in Drosophila brain, is involved in the catabolism of monoamines such as tryptamine, tyramine, serotonin, dopamine, and octopamine (Hintermann et al., Proc. Natl. Acad. Sci. USA 93:12315 (1996); Brodbeck et al., DNA Cell Biol.17:621(1998)). In rats, waking is associated with a marked increase in brain mRNA for arylsulfotransferase, another enzyme implicated in the catabolism of monoamines (Cirelli et al., Mol. Brain Res. 56, 293 (1998); Cirelli et al., Ann. Med. 31:117 (1999); Cirelli et al., Sleep 22(S):113 (1999)). These findings are of importance because, in the species tested so far, waking is associated with high central monoaminergic activity, while a reduction of such activity is a hallmark of sleep (McGinty et al., Brain Res. 101: 569 (1976); Aston-Jones et al., 1:876 (1981)). This has led to the suggestion that sleep may serve to counteract the effects of continued monoaminergic discharge. According to this hypothesis, an impaired catabolism of monoamines should result in an increased need for sleep (Hartmann et al., Functions of Sleep, (Yale University Press, New Haven (1973); Siegel et al., Brain Res. Rev. 13:213 (1988); Jouvet, Neuropsychopharm. 21, 24S (1999)).

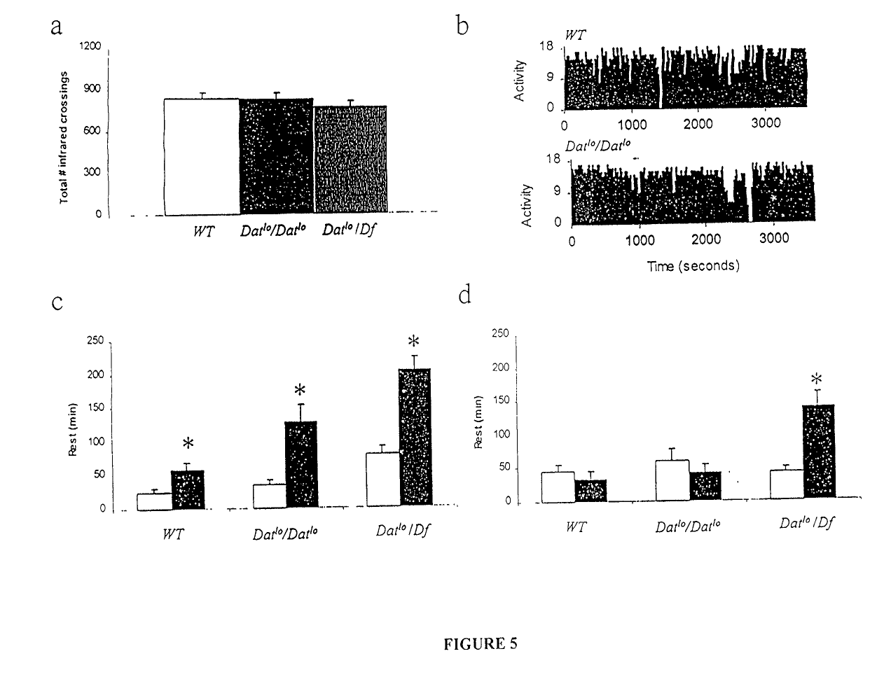

To evaluate this possibility, a Drosophila mutant was used in which the activity of the Dat enzyme is deficient (Dat^{lo}). Dat^{lo} is a hypomorphic allele of AANATlb. Insertion of blastopia into the first intron results in 10% of wildtype dopamine acetyltransferase activity. As indicated by both the infrared and ultrasound monitoring systems, flies homozygous for the Dat^{lo} mutation did not differ from wild-types in the percentage and circadian distribution of rest and waking under baseline conditions (FIG. 5A). They also showed normal amounts and patterns of activity (FIG. 5B). Each strain obtained >90% of their daily rest during the dark period. However, following 12 h of rest deprivation during the dark period, it was found that Dat^{lo} flies displayed a rest rebound that was much greater than in rest deprived controls (189%) (FIG. 5C).

To confirm that this phenotype maps to the Dat locus and to assay for gene dosage effects, flies with one dose of the Dat^{lo} mutation (hemizygous) were generated by crossing Dat^{lo} homozygotes with flies carrying a deficiency (Df) of the Dat locus, Df(2R)Px1. Flies hemizygous for the Dat^{lo} mutation (Dat^{lo}/Df) did not differ from wild-types or Dat^{lo} homozygotes in the percentage and circadian distribution of rest and waking under baseline conditions (FIG. 5A). Dat^{lo}/Df flies showed not only an increased rest rebound during the first 6 h of recovery compared to wild-type flies (FIG. 5C), but also a persistent rebound during the second 6 h of recovery (FIG. 5D). These results indicate that the more severely mutant the fly is at the Dat locus, the greater the rebound. Although the mechanism responsible for the increased homeostatic response to rest deprivation is not clear, these results suggest a linkage between the catabolism of monoamines and the regulation of sleep and waking in Drosophila.

In order to evaluate whether other genes involved in monoamine catabolism are associated with altered vigilance, mutants in Dopa decaryboxylase (Ddc) were evaluated. Dopa decaryboxylase (Ddc) is involved in the final step in the synthesis of the neurotransmitter dopamine. Two genotypes, Ddc[ts2]/+ and Ddc[27]/+, both heterozygous for Ddc mutations, were tested. Ddc[ts2]/+ has somewhat more enzyme activity than Ddc[27]/+. Ddc[ts2]/+ and Ddc[27]/+ Drosophila were tested initially for activity and sleep, both of which were normal. Ddc[ts2]/+ and Ddc[27]/+ Drosophila were then tested for rebound effect after sleep deprivation. Both Ddc[ts2]/+ and Ddc[27]/+ Drosophila exhibited approximately half as much rebound as wild-type flies. Moreover, the rebound in Ddc[27]/+ flies (2 hr long) was shorter than in Ddc[ts2]/+ flies(4 hr long), as compared to wild-type (6 hr long). These results are consistent with a role for Ddc in homeostatic regulation of sleep. More specifically, the less Ddc enzyme activity, the less rebound.

The results observed with Ddc mutants are also consistent with the Dat results. Dat mutants fail to degrade several neurotransmitters, including dopamine. The less Dat activity the flies have, the more and longer rebound they show. The Ddc mutants exhibit opposite behavior—the less neurotransmitter produced, the less rebound. Thus, there is an apparent correlation between the accumulation of neurotransmitters such as dopamine and the amount of rebound.

Taken together, the results shown in Examples I-IV indicate that rest in invertebrates is very similar to mammalian sleep, as evidenced by increased arousal threshold, homeostatic regulation, dependence on age, sensitivity to pharmacological manipulation, and expression of similar vigilance-modulated genes.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention. Accordingly, the invention is limited only by the claims.

We claim:

1. An isolated vigilance nucleic acid molecule, comprising a nucleotide sequence selected from the group consisting of SEQ ID NOS:1-6 and 8-27, or modification thereof.

2. The isolated nucleic acid molecule of claim 1, attached to a solid support.

3. An isolated oligonucleotide, comprising at least 15 contiguous nucleotides of the nucleotide sequence of SEQ ID NOS:1-6 and 8-27, or the antisense strand thereof.

4. The isolated oligonucleotide of claim 3, attached to a solid support.

5. A kit, comprising two or more isolated oligonucleotides according to claim 3.

6. The kit of claim 5, comprising a PCR primer pair.

7. A kit, comprising two or more isolated vigilance nucleic acid molecules, wherein at least one vigilance nucleic acid molecule comprises a nucleotide sequence selected from the group consisting of SEQ ID NOS:1-6 and 8-27 or modification thereof.

8. The kit of claim 7, wherein said isolated vigilance nucleic acid molecules are attached to a solid support.

9. The kit of claim 7, further comprising one or more isolated nucleic acid molecules selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, NGFI-A, NGFI-B, rlf, Arc, JunB, IER5, Cytochrome oxidase C subunit 1, Cytochrome oxidase C subunit IV, NADH dehydrogenase subunit 2, 12S rRNA F1-ATPase subunit alpha, Ng/RC3, bone morphogenetic protein 2, GRP78, BDNF, IL-1β, dendrin, Ca⁺⁺/calmodulin-dependent protein kinase II α-subunit, orexin, orexin receptor, and PRNP.

10. A method of diagnosing a vigilance disorder in an individual, comprising:
determining a vigilance gene profile of the individual, and
comparing said profile to a control profile indicative of the vigilance disorder, wherein at least one vigilance gene profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene comprising a nucleotide sequence of any of SEQ ID NOS:2-6, 8-14 and 16-27 or modification thereof,
wherein correspondence between said profile of said individual and said control profile indicates that said individual has said vigilance disorder.

11. A method of determining vigilance level in an individual, comprising:
determining a vigilance gene profile of the individual, and
comparing said profile to a control profile indicative of a predetermined vigilance level, wherein at least one vigilance gene profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene comprising a nucleotide sequence of any of SEQ ID NOS:2-6, 8-14 and 16-27 or modification thereof,
wherein correspondence between said profile of said individual and said control profile indicates that said individual exhibits said vigilance level.

12. A method of determining the efficacy of a compound in ameliorating a vigilance disorder, comprising:
administering the compound to an individual having a vigilance disorder, and
determining an effect of the compound on the vigilance gene profile of the individual, wherein at least one vigilance gene profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene comprising a nucleotide sequence of any of SEQ ID NOS:2-6, 8-14 and 16-27 or modification thereof,
wherein modulation of the vigilance gene profile of the individual to correspond to a normal vigilance profile indicates that the compound is effective in ameliorating the vigilance disorder.

13. A method of determining the efficacy of a compound in modulating vigilance, comprising:
administering the compound to an individual, and
determining an effect of the compound on the vigilance gene profile of the individual, wherein at least one vigilance gene profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene comprising a nucleotide sequence of any of SEQ ID NOS:2-6, 8-14 and 16-27 or modification thereof,
wherein modulation of the vigilance gene profile indicates that the compound modulates vigilance.

14. A method of ameliorating a vigilance disorder in an individual, comprising:
administering to an individual having a vigilance disorder an agent that modulates the vigilance gene profile of the individual to correspond to a normal vigilance gene profile,
wherein at least one vigilance gene profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene comprising a nucleotide sequence of any of SEQ ID NOS:2-6, 8-14 and 16-27 or modification thereof.

15. A method of modulating vigilance level in an individual, comprising:
administering to an individual an agent that modulates the vigilance gene profile of the individual to correspond to a control vigilance gene profile,
wherein at least one vigilance gene profiled is selected from the group consisting of Fas, BiP, Cyp4e2, AANAT1 (Dat), Ddc, Cytochrome P450, AA117313, aryl sulfotransferase IV, human breast tumor autoantigen homolog, KIAA313 homolog, E25, and a gene comprising a nucleotide sequence of any of SEQ ID NOS:2-6, 8-14 and 16-27 or modification thereof.
